(19) United States
(12) Patent Application Publication
(10) Pub. No.: US 2025/0145630 A1
(43) Pub. Date: May 8, 2025

(54) POLYMORPHS OF A PYRIDINYLIMIDAZO[1,2-B]PYRIDAZINE

(71) Applicant: Cardurion Pharmaceuticals, Inc., Burlington, MA (US)

(72) Inventors: John Amedio, Burlington, MA (US); Tomohiro Okawa, Kanagawa (JP); Junya Shirai, Kanagawa (JP); Erika Volckova, Burlington, MA (US)

(21) Appl. No.: 18/939,305

(22) Filed: Nov. 6, 2024

Related U.S. Application Data
(60) Provisional application No. 63/596,736, filed on Nov. 7, 2023.

(51) Int. Cl. C07D 487/04; C07C 57/15; C07C 59/255; C07C 309/04

(52) U.S. Cl. CPC C07D 487/04; C07C 57/15; C07C 59/255; C07C 309/04; C07B 2200/13

(57) ABSTRACT

The present application relates to salts, co-crystals, and polymorphs of (S)-2-(6-(3-((1-(1H-tetrazol-1-yl)propan-2-yl)oxy)-4-fluorophenyl)imidazo[1,2-b]pyridazin-3-yl)-4-methoxynicotinonitrile (Formula I), and compositions and uses thereof.

### CROSS-REFERENCE TO RELATED APPLICATION

This application claims priority to U.S. Provisional Application No. 63/596,736, filed Nov. 7, 2023, which is incorporated herein in its entirety for all purposes.

### BACKGROUND OF THE INVENTION

Cardiac diseases include heart failure, arrhythmia, myocardial infarction, angina, valvular heart disease and the like, and they are high-mortality diseases. In treatment of cardiac diseases with a drug, the symptoms are improved by control of each risk factor and symptomatic therapy. However, the satisfaction with treatment remains low level, and there is now no definitive therapy.

Calcium-calmodulin complex binds to Ca²⁺/calmodulin-dependent protein kinase (CaMK) included in serine/threonine protein kinase, and activates the kinase. The CaMK family includes CaMKII, and four isoforms (α, β, γ and δ) exist as CaMKII. CaMKII α and CaMKII β are expressed mainly in cerebral tissue, and CaMKII γ and CaMKII δ are expressed in many tissues including heart. CaMKII is activated by amino acid-modification due to oxidative stress or hyperglycemia, in addition to the binding of calcium-calmodulin complex. CaMKII regulates cell functions by phosphorylation of a transcription factor which is a substrate, a protein that plays a function in organelle uptake/excretion of Ca²⁺, a protein that regulates contract and relax of muscles, a channel that regulates an intracellular ion concentration, and the like, due to its kinase activation.

Some documents suggest that CaMKII plays a harmful role in progress of cardiac disease conditions. Expression and activity of CaMKII are increased in heart of human patient or animal with heart failure. In transgenic mouse overexpressing CaMKII δ in heart, onsets of cardiac hypertrophy and heart failure are reported. By studies using an inhibitor by a pharmacological method, and studies using a gene deletion by genetic method, protecting effects on heart failure, cardiac hypertrophy, myocardial infarction and arrhythmia by an inhibition of CaMKII and an overexpression of CaMKII inhibitory protein are reported in mouse. For catecholaminergic polymorphic ventricular tachycardia, improving effects on disease conditions by CaMKII inhibitor in mutant ryanodine knock-in mouse (RyR2^{R4496C+/-} mouse) are reported. These findings suggest availabilities of CaMKII inhibitors in the prophylaxis and/or treatment of cardiac diseases including heart failure, cardiac hypertrophy, myocardial infarction and cardiac arrhythmia.

Recently, CaMKII exacerbating action on growth or metastasis of a certain type of cancer is suggested. In addition, therapeutic effect on acute renal failure, intimal hypertrophy, hepatic fibrosis, stroke, pain, rheumatoid arthritis and the like by CaMKII inhibition are also indicated.

However, genetic methods achieve only deficiency of protein or overexpression of inhibitory protein, and they are different from a mechanism which inhibits temporarily kinase activity, and therefore, effects by kinase inhibitor cannot be always expected. In addition, inhibitors which have been already reported are not suitable for application as a medicament for a CaMKII selective inhibitor, because they have a low kinase selectivity to CaMKII, or they are not suitable for oral administration or chronic administration.

(S)-2-(6-(3-((1-(1H-tetrazol-1-yl)propan-2-yl)oxy)-4-fluorophenyl)imidazo[1,2-b]pyridazin-3-yl)-4-methoxynicotinonitrile has been described as the CaMKII inhibitor Example 321 (IC₅₀<10 nM) in US publication no. 20220098207 on pages 74 and 96.

### BRIEF SUMMARY OF THE INVENTION

In some embodiments, the present invention provides a crystalline form of the compound of Formula (I):

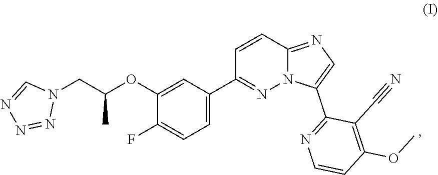 [SMILES: COc1ccnc(-c2cnc3ccc(-c4ccc(F)c(O[C@@H](C)Cn5cnnn5)c4)nn23)c1C#N]

or a pharmaceutically acceptable salt thereof.

In some embodiments, a pharmaceutical composition of the present disclosure comprises a crystalline form as described herein, and a pharmaceutically acceptable excipient.

In some embodiments, a method of the present disclosure is a method of preparing Form C of the compound of Formula (I), comprising: (a) warming a mixture of the compound of Formula (I) and a solvent comprising acetone, water, dichloromethane, or methyl ethyl ketone, or a combination thereof, and (b) cooling the mixture, thereby preparing the Form C of the compound of Formula (I).

In some embodiments, a method of the present disclosure is a method of preventing or treating a CaMKII associated disease or condition, comprising administering a therapeutically effective amount of a crystalline form as described herein.

### BRIEF DESCRIPTION OF THE DRAWINGS

FIG. 1 shows an X-ray powder diffraction (XRPD) pattern for Form A of the compound of Formula (I).

FIG. 2 shows a differential scanning calorimetry (DSC) graph for Form A of the compound of Formula (I).

FIG. 3 shows an X-ray powder diffraction (XRPD) pattern for Form B of the compound of Formula (I).

FIG. 4 shows an X-ray powder diffraction (XRPD) pattern for Form C of the compound of Formula (I).

FIG. 5 shows thermogravimetric analysis (TGA)/DSC graph of Form C of the compound of Formula (I).

FIG. 6 shows a differential scanning calorimetry (DSC) of Form C of the compound of Formula (I).

FIG. 7 shows a dynamic vapor sorption (DVS) of Form C of the compound of Formula (I).

FIG. 8 shows an X-ray powder diffraction (XRPD) pattern for the L-tartaric acid co-crystal Form 5 of the compound of Formula (I).

FIG. 9 shows an X-ray powder diffraction (XRPD) pattern for the L-tartaric acid co-crystal Form 1 of the compound of Formula (I).

FIG. 10 shows an X-ray powder diffraction (XRPD) pattern for the L-tartaric acid co-crystal Form 2 of the compound of Formula (I).

FIG. 11 shows a TGA/DSC of the L-tartaric acid co-crystal Form 2 of the compound of Formula (I).

FIG. 12 shows a differential scanning calorimetry (DSC) of the L-tartaric acid co-crystal Form 2 of the compound of Formula (I). The DSC was obtained by heating at 1° C./min.

FIG. 13 shows a dynamic vapor sorption (DVS) of the L-tartaric acid co-crystal Form 2 of the compound of Formula (I).

FIG. 14 shows an X-ray powder diffraction (XRPD) pattern for the L-tartaric acid co-crystal Form 3 of the compound of Formula (I).

FIG. 15 shows an X-ray powder diffraction (XRPD) pattern for a fumaric acid co-crystal of the compound of Formula (I).

FIG. 16 shows an X-ray powder diffraction (XRPD) pattern for a hydrochloride salt of the compound of Formula (I).

FIG. 17 shows an X-ray powder diffraction (XRPD) pattern for a methanesulfonate salt of the compound of Formula (I).

FIG. 18 shows a TGA/DSC of a methanesulfonate salt of the compound of Formula (I).

FIG. 19 shows a differential scanning calorimetry (DSC) of a methanesulfonate salt of the compound of Formula (I).

FIG. 20 shows a dynamic vapor sorption (DVS) of a methanesulfonate salt of the compound of Formula (I).

FIG. 21 shows an X-ray powder diffraction (XRPD) pattern for a phosphoric acid co-crystal of the compound of Formula (I).

### DETAILED DESCRIPTION OF THE INVENTION

### I. General

The compound (S)-2-(6-(3-((1-(1H-tetrazol-1-yl)propan-2-yl)oxy)-4-fluorophenyl)imidazo[1,2-b]pyridazin-3-yl)-4-methoxynicotinonitrile (Formula I) is a selective and potent inhibitor of calcium-calmodulin (CaM)-dependent protein kinase II (CaMKII):

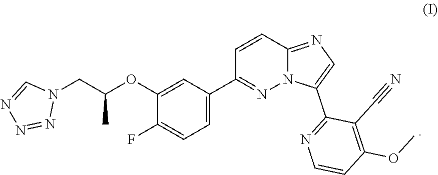 [SMILES: COc1ccnc(-c2cnc3ccc(-c4ccc(F)c(O[C@@H](C)Cn5cnnn5)c4)nn23)c1C#N]

The present invention results from the unexpected results of the solid forms of Formula I or pharmaceutically acceptable salt thereof, advantages attributed to the forms as described herein, and processes for making the solid forms. Crystalline materials can be more stable physically and chemically. The superior stability of crystalline material may make it more suitable to be used in the final dosage form as shelf life of the product is directly correlated with stability. A crystallization step in API processing also means an opportunity to upgrade the drug substance purity by rejecting the impurities to the processing solvent.

### II. Definitions

Unless defined otherwise, all technical and scientific terms used herein have the same meaning as commonly understood by one of ordinary skill in the art.

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3.

A “crystalline form of the disclosure” includes crystalline forms described herein, for example a crystalline form of the disclosure includes crystalline forms of Formula (I) or a pharmaceutically acceptable salt or co-crystal thereof, including the crystalline forms of the Examples.

“Solvate” refers to a complex formed by the combining of the crystalline form of Formula I and a solvent.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier understood in the art. In some embodiments, a pharmaceutically acceptable excipient has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In some embodiments, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” refers to an amount that is sufficient to effect treatment, as defined below, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Subject” refers to animals such as mammals, including, but not limited to, primates (e.g., humans), cows, sheep, goats, horses, dogs, cats, rabbits, rats, mice and the like. In some embodiments, the subject is a human. In some embodiments, the subject is a patient.

“Disease” or “condition” refer to a state of being or health status of a subject capable of being treated with a crystalline form, pharmaceutical composition, or method provided herein.

### III. Crystalline Forms

The present disclosure describes, inter alia, a crystalline form of the compound of Formula (I):

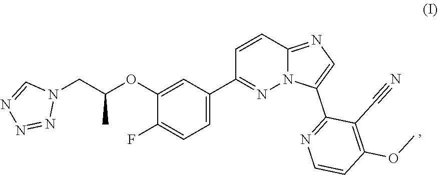 [SMILES: COc1ccnc(-c2cnc3ccc(-c4ccc(F)c(O[C@@H](C)Cn5cnnn5)c4)nn23)c1C#N]

or a pharmaceutically acceptable salt thereof.

In some embodiments, a crystalline form of the compound of Formula (I) or pharmaceutically acceptable salt thereof is superior in a physical and/or chemical property compared with a non-crystalline form of the compound. Crystalline materials can be more stable physically and chemically. A superior stability of a crystalline material may make it more suitable to be used in the final dosage form as shelf life of the product is directly correlated with stability. A crystallization step in processing of the active ingredient also means an opportunity to upgrade the drug substance purity by rejecting the impurities to the processing solvent.

Accordingly, in some embodiments, a crystalline form of the compound of Formula (I) or pharmaceutically acceptable salt thereof is stable under extended storage conditions, e.g., longer than about 1 month, about 3 months, about 6 months, about 1 year, about 2 years, about 3 years, or longer. In some embodiments, the extended storage conditions comprises storage at about 25° C. and about 60% relative humidity (RH). In some embodiments, the extended storage conditions comprises storage at about 40° C. and about 75% relative humidity (RH). In some embodiments, a crystalline form of the compound of Formula (I) or pharmaceutically acceptable salt thereof has from about 95% to about 100% purity, e.g., greater than about 95%, about 96%, about 98%, about 99%, about 99.2%, about 99.5%, about 99.6%, about 99.7%, about 99.8%, about 99.9%, about 99.99% purity.

In some embodiments, a crystalline form of the compound of Formula (I) or pharmaceutically acceptable salt thereof is superior in vivo kinetics (e.g., plasma drug half-life, intracerebral transferability, metabolic stability) and/or shows low toxicity (e.g., superior as a medicament in terms of liver/hepatotoxicity, acute toxicity, chronic toxicity, genetic toxicity, reproductive toxicity, cardiotoxicity, cytotoxicity, drug interaction, carcinogenicity etc.; especially liver/hepatotoxicity) compared with a non-crystalline form of the compound.

In some embodiments, the crystalline form is a pharmaceutically acceptable salt of the compound of Formula (I). In some embodiments, the pharmaceutically acceptable salt is a salt recognized in the art. For example, in some embodiments, the pharmaceutically acceptable salt is one described in Remington: The Science and Practice of Pharmacy, 23th edition, Adejare, A., ed. Academic Press: London, 2020. In some embodiments, a salt of the compound of Formula (I) results if the compound of Formula (I) and an acid have a ΔpKa (pKa (conjugate acid of base)−pKa (acid))>1, such that a substantial proton transfer results in ionization and formation of the salt. In some embodiments, the compound of Formula (I) and an acid have a ΔpKa (pKa (conjugate acid of base)−pKa (acid))>1.

In some embodiments, the crystalline form is the form wherein the pharmaceutically acceptable salt is a hydrochloride, hydrobromide, sulfate, methanesulfonate, ethanesulfonate, 2-hydroxyethanesulfonate, camphorsulfonate, besylate, tosylate, naphthalene-2-sulfonate, naphthalene-1,5-disulfonate, or ethane-1,2-disulfonate.

In some embodiments, the crystalline form is a co-crystal of the compound of Formula (I). As generally understood in the art, co-crystals can be distinguished from salts because unlike salts, the components that co-exist in the co-crystal lattice with a defined stoichiometry interact nonionically. See, e.g., U.S. Department of Health and Human Services, Food and Drug Administration Center for Drug Evaluation and Research. Regulatory Classification of Pharmaceutical Co-Crystals Guidance for Industry, February 2018. In some embodiments, in the co-crystal, the second component of the lattice, the coformer, is not a solvent. In some embodiments, the coformer is nonvolatile. In some embodiments, a co-crystal of the compound of Formula (I) is formed if the compound of Formula (I) and the coformer have a ΔpKa (pKa (conjugate acid of base)−pKa (acid))<1. In some embodiments, the co-crystal is a tartaric acid, fumaric acid, or phosphoric acid co-crystal.

In some embodiments, the crystalline form is a free base of the compound of Formula (I). In some embodiments, the crystalline form is anhydrous.

### Form A

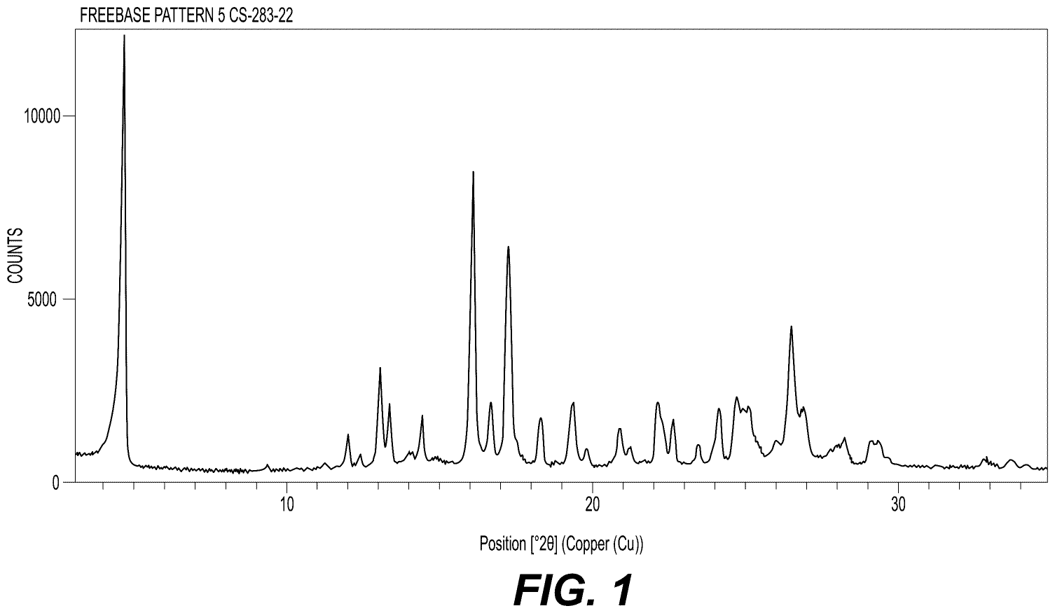

In some embodiments, the crystalline form is Form A. In some embodiments, the Form A is characterized by an X-ray powder diffraction (XRPD) pattern comprising peaks at 4.7, 16.1, and 26.5 degrees 2θ (±0.2 degrees 2θ), wherein the XRPD is made using CuK_{α1} radiation. In some embodiments, the Form A is characterized by an X-ray powder diffraction (XRPD) pattern comprising peaks at 4.7, 16.1, 17.2, and 26.5 degrees 2θ (±0.2 degrees 2θ), wherein the XRPD is made using CuK_{α1} radiation. In some embodiments, the Form A is characterized by an X-ray powder diffraction (XRPD) pattern comprising peaks at 4.7, 13.1, 16.1, 17.2, and 26.5 degrees 2θ (±0.2 degrees 2θ), wherein the XRPD is made using CuK_{α1} radiation. In some embodiments, the Form A is characterized by an X-ray powder diffraction (XRPD) pattern comprising peaks as shown in Table 1, wherein the XRPD is made using CuK_{α1} radiation. In some embodiments, the Form A is characterized by an X-ray powder diffraction (XRPD) pattern substantially as shown in FIG. 1, wherein the XRPD is made using CuK_{α1} radiation.

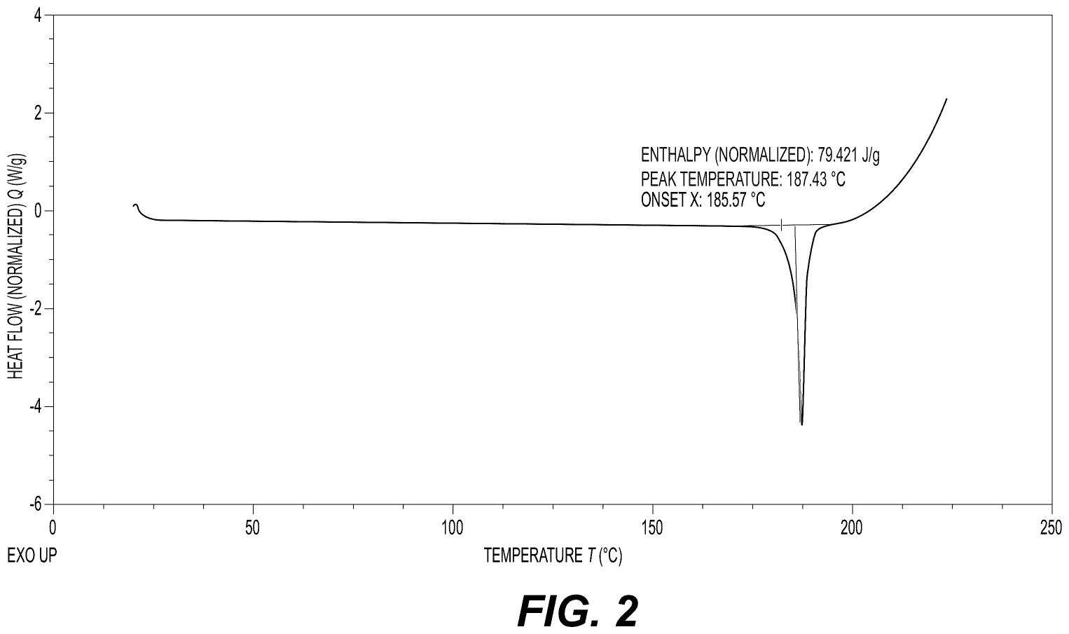

In some embodiments, the Form A is characterized by a differential scanning calorimetry (DSC) graph having an endotherm at about 187° C. In some embodiments, the Form A is characterized by a differential scanning calorimetry (DSC) graph substantially as shown in FIG. 2.

### Form B

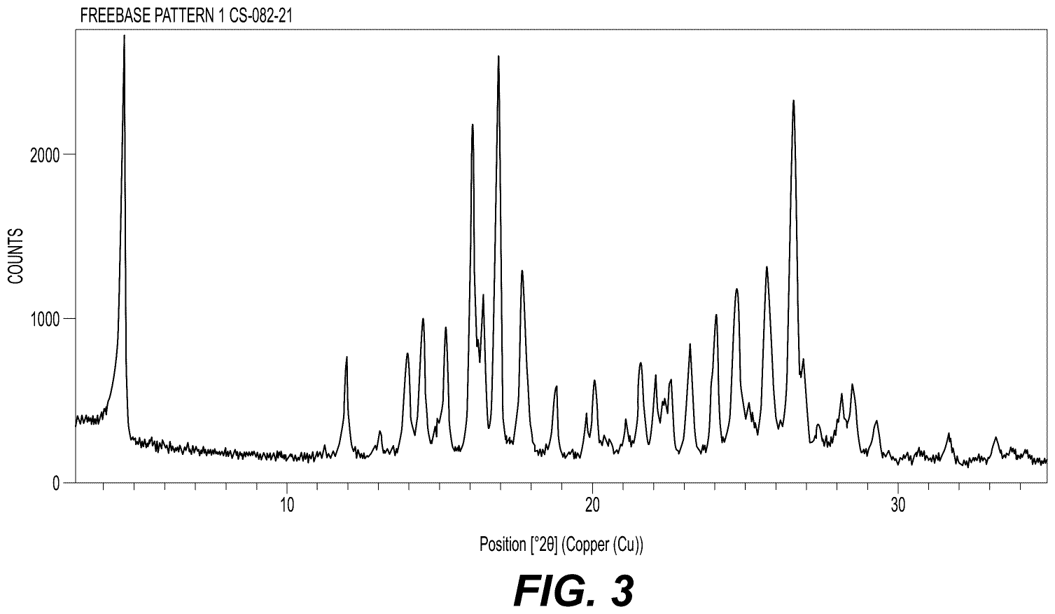

In some embodiments, the crystalline form is Form B. In some embodiments, the Form B is characterized by an X-ray powder diffraction (XRPD) pattern comprising peaks at 16.1, 16.9, and 26.6 degrees 2θ (±0.2 degrees 2θ), wherein the XRPD is made using CuK_{α1} radiation. In some embodiments, the Form B is characterized by an X-ray powder diffraction (XRPD) pattern comprising peaks as shown in Table 2, wherein the XRPD is made using CuK_{α1} radiation. In some embodiments, the Form B is characterized by an X-ray powder diffraction (XRPD) pattern substantially as shown in FIG. 3, wherein the XRPD is made using CuK_{α1} radiation.

### Form C

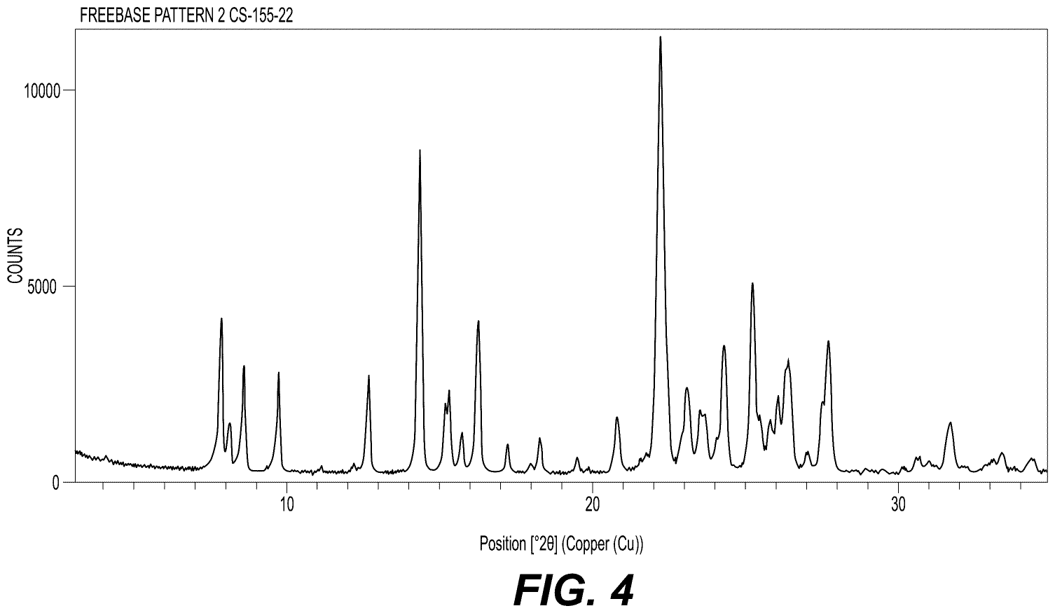

In some embodiments, the crystalline form is Form C. In some embodiments, the crystalline form is anhydrous. In some embodiments, the Form C is characterized by an X-ray powder diffraction (XRPD) pattern comprising peaks at 14.4, 22.2, and 25.2 degrees 2θ (±0.2 degrees 2θ), wherein the XRPD is made using CuK_{α1} radiation. In some embodiments, the Form C is characterized by an X-ray powder diffraction (XRPD) pattern comprising peaks at 14.4, 16.3, 22.2, and 25.2 degrees 2θ (±0.2 degrees 2θ), wherein the XRPD is made using CuK_{α1} radiation. In some embodiments, the Form C is characterized by an X-ray powder diffraction (XRPD) pattern comprising peaks at 14.4, 16.3, 22.2, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 14.4, 16.3, 22.2, 24.3, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 8.6, 14.4, 16.3, 22.2, 24.3, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 8.6, 9.7, 14.4, 16.3, 22.2, 24.3, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 7.8, 8.6, 9.7, 14.4, 16.3, 22.2, 24.3, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 7.8, 8.1, 8.6, 9.7, 12.7, 14.4, 16.3, 22.2, 25.2, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks at 7.8, 8.6, 9.7, 12.7, 14.4, 16.3, 22.2, 24.3, 25.2, 26.1, 26.4, and 27.7 degrees 2θ (±0.2 degrees 2θ), wherein the XRPD is made using CuK_{α1} radiation. In some embodiments, the Form C is characterized by an X-ray powder diffraction (XRPD) pattern comprising peaks as shown in Table 3, wherein the XRPD is made using CuK_{α1} radiation. In some embodiments, the Form C is characterized by an X-ray powder diffraction (XRPD) pattern substantially as shown in FIG. 4, wherein the XRPD is made using CuK_{α1} radiation.

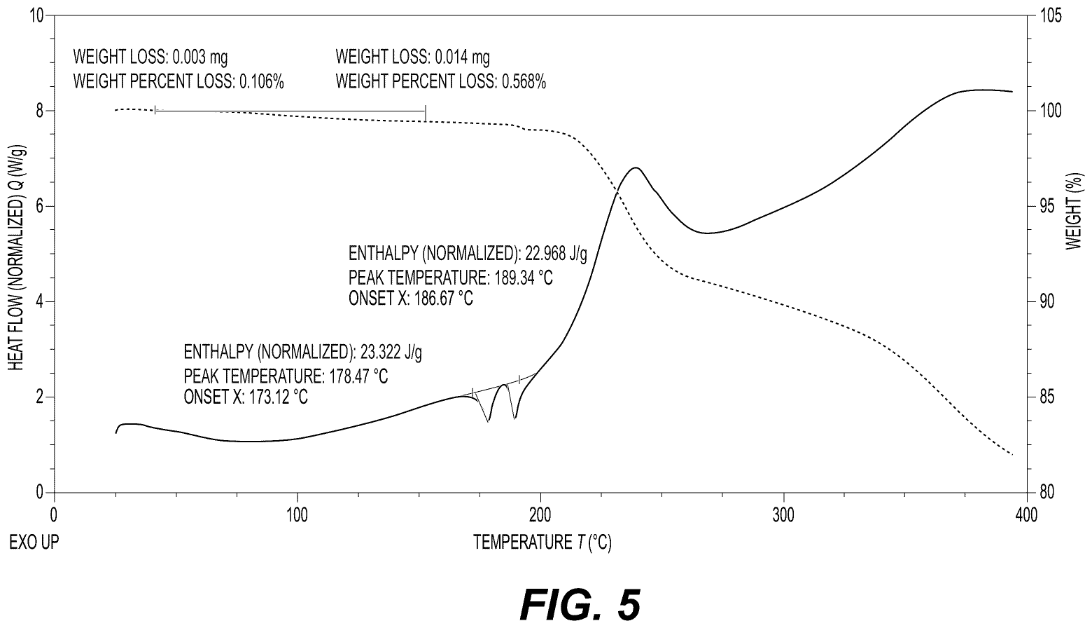

In some embodiments, the Form C is characterized by a thermogravimetric analysis (TGA) graph substantially as shown in FIG. 5.

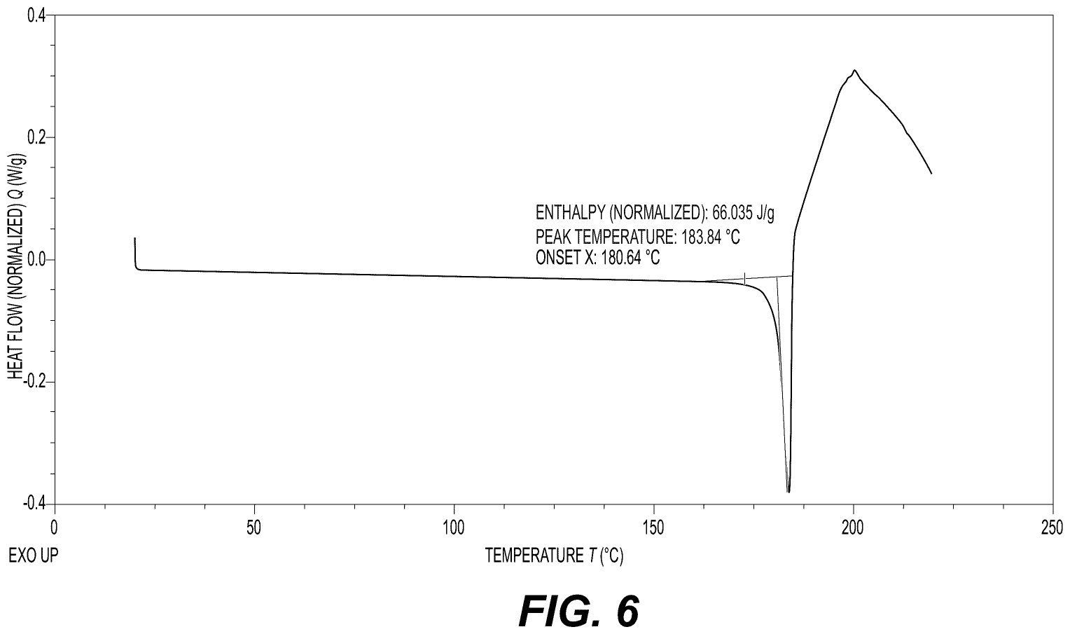

In some embodiments, the Form C is characterized by a differential scanning calorimetry (DSC) graph having an endothermic peak at about 184° C. In some embodiments, the Form C is characterized by a differential scanning calorimetry (DSC) graph substantially as shown in FIG. 6.

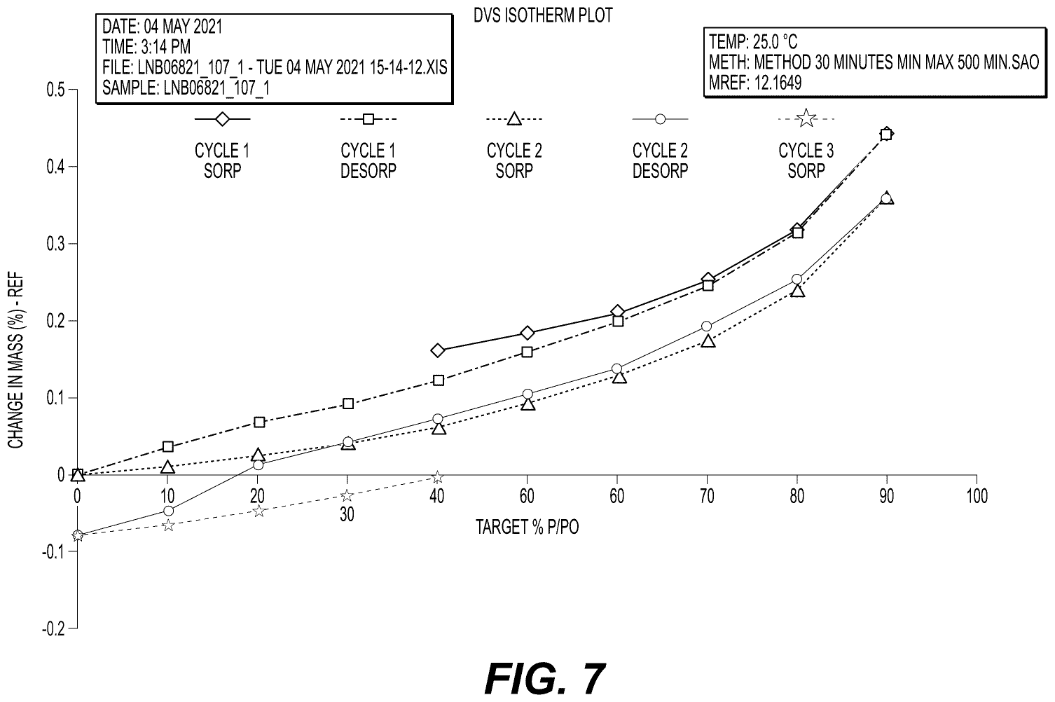

In some embodiments, the Form C is characterized by a dynamic vapor sorption (DVS) plot substantially as shown in FIG. 7.

In some embodiments, Form C of the compound of Formula (I) is stable under extended storage conditions, e.g., longer than about 1 month, about 3 months, about 6 months, about 1 year, about 2 years, about 3 years, or longer. In some embodiments, the extended storage conditions comprises storage at about 25° C. and about 60% relative humidity (RH). In some embodiments, the extended storage conditions comprises storage at about 40° C. and about 75% relative humidity (RH). In some embodiments, Form C of the compound of Formula (I) has from about 95% to about 100% purity, e.g., greater than about 95%, about 96%, about 98%, about 99%, about 99.2%, about 99.5%, about 99.6%, about 99.7%, about 99.8%, about 99.9%, about 99.99% purity.

### Tartaric Acid Co-Crystals

In some embodiments, the crystalline form is a co-crystal of the compound of Formula (I). In some embodiments, the crystalline form is a tartaric acid co-crystal. In some embodiments, the crystalline form is a L-tartaric acid co-crystal. In some embodiments, the crystalline form is a co-crystal having about 1:1 ratio of the compound of Formula (I) and L-tartaric acid. In some embodiments, the crystalline form is a co-crystal having about 1:1:1 ratio of the compound of Formula (I), L-tartaric acid, and water.

### Tartaric Acid Form 5

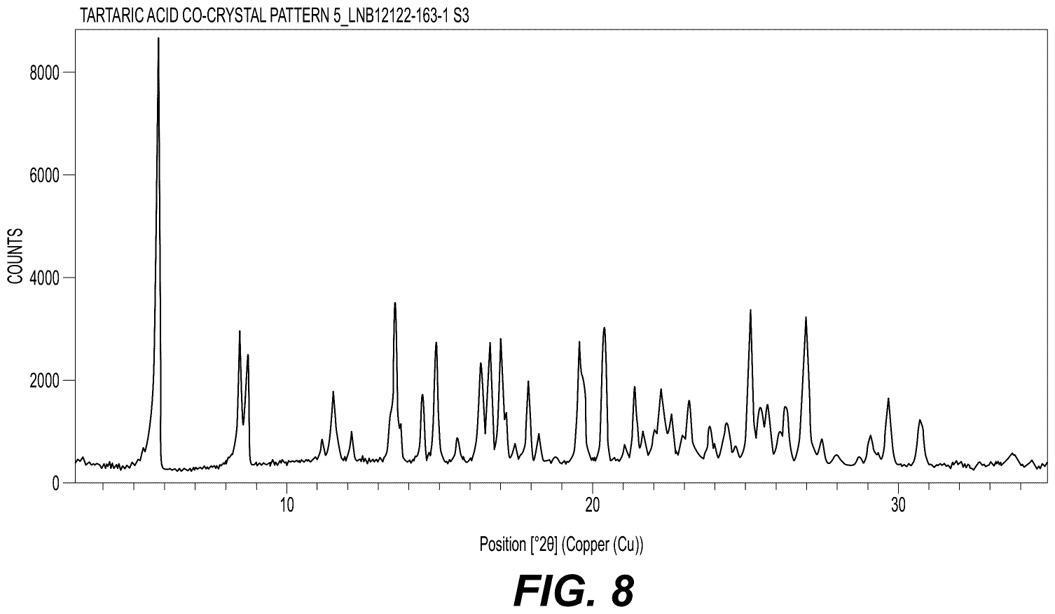

In some embodiments, the crystalline form is L-tartaric acid co-crystal Form 5. In some embodiments, the L-tartaric acid co-crystal Form 5 is characterized by an X-ray powder diffraction (XRPD) pattern comprising peaks at 5.8, 8.5, and 13.5 degrees 2θ (±0.2 degrees 2θ), wherein the XRPD is made using CuK_{α1} radiation. In some embodiments, the L-tartaric acid co-crystal Form 5 is characterized by an X-ray powder diffraction (XRPD) pattern comprising peaks at 5.8, 8.5, 13.5, and 25.1 degrees 2θ (±0.2 degrees 2θ), wherein the XRPD is made using CuK_{α1} radiation. In some embodiments, the L-tartaric acid co-crystal Form 5 is characterized by an X-ray powder diffraction (XRPD) pattern comprising peaks at 5.8, 8.5, 13.5, 25.1, and 27.0 degrees 2θ (±0.2 degrees 2θ), wherein the XRPD is made using CuK_{α1} radiation. In some embodiments, the L-tartaric acid co-crystal Form 5 is characterized by an X-ray powder diffraction (XRPD) pattern comprising peaks at 5.8, 8.5, 13.5, 17.0, 25.1, and 27.0 degrees 2θ (±0.2 degrees 2θ), wherein the XRPD is made using CuK_{α1} radiation. In some embodiments, the L-tartaric acid co-crystal Form 5 is characterized by an X-ray powder diffraction (XRPD) pattern comprising peaks at 5.8, 8.5, 13.5, 17.0, 19.6, 25.1, and 27.0 degrees 2θ (±0.2 degrees 2θ), wherein the XRPD is made using CuK_{α1} radiation. In some embodiments, the L-tartaric acid co-crystal Form 5 is characterized by an X-ray powder diffraction (XRPD) pattern comprising peaks as shown in Table 9, wherein the XRPD is made using CuK_{α1} radiation. In some embodiments, the L-tartaric acid co-crystal Form 5 is characterized by an X-ray powder diffraction (XRPD) pattern substantially as shown in FIG. 8, wherein the XRPD is made using CuK_{α1} radiation.

### Tartaric Acid Form 1

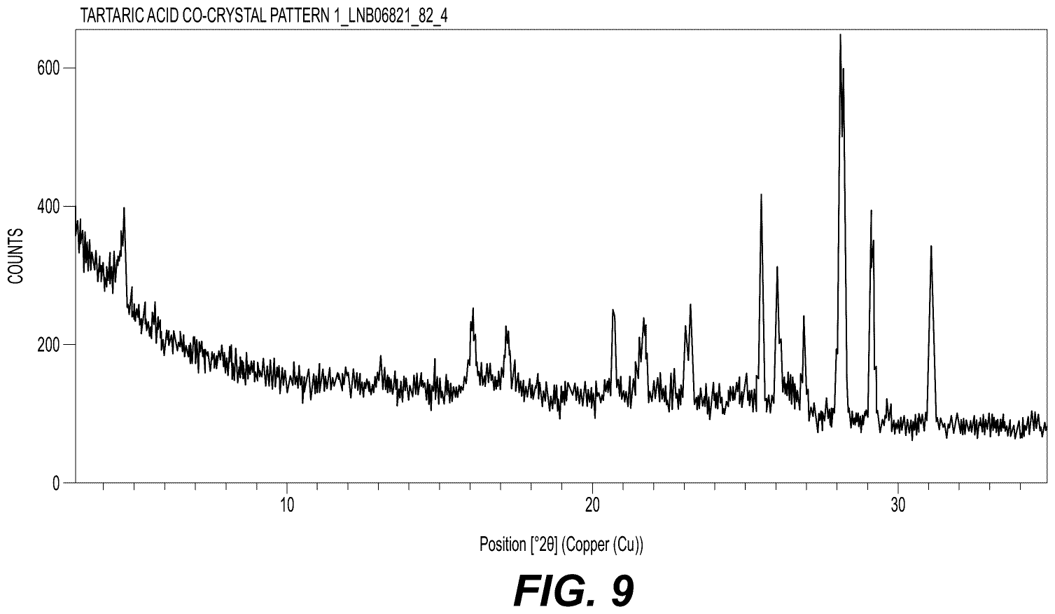

In some embodiments, the crystalline form is L-tartaric acid co-crystal Form 1. In some embodiments, the L-tartaric acid co-crystal Form 1 is characterized by an X-ray powder diffraction (XRPD) pattern comprising peaks at 25.5, 28.1, and 29.1 degrees 2θ (±0.2 degrees 2θ), wherein the XRPD is made using CuK_{α1} radiation. In some embodiments, the L-tartaric acid co-crystal Form 1 is characterized by an X-ray powder diffraction (XRPD) pattern comprising peaks at 25.5, 28.1, 29.1, and 31.1 degrees 2θ (±0.2 degrees 2θ), wherein the XRPD is made using CuK_{α1} radiation. In some embodiments, the L-tartaric acid co-crystal Form 1 is characterized by an X-ray powder diffraction (XRPD) pattern comprising peaks at 25.5, 26.1, 28.1, 29.1, and 31.1 degrees 2θ (±0.2 degrees 2θ), wherein the XRPD is made using CuK_{α1} radiation. In some embodiments, the L-tartaric acid co-crystal Form 1 is characterized by an X-ray powder diffraction (XRPD) pattern comprising peaks at 23.2, 25.5, 26.1, 28.1, 29.1, and 31.1 degrees 2θ (±0.2 degrees 2θ), wherein the XRPD is made using CuK_{α1} radiation. In some embodiments, the L-tartaric acid co-crystal Form 1 is characterized by an X-ray powder diffraction (XRPD) pattern comprising peaks at 4.7, 23.2, 25.5, 26.1, 26.9, 28.1, 29.1, and 31.1 degrees 2θ (±0.2 degrees 2θ), wherein the XRPD is made using CuK_{α1} radiation. In some embodiments, the L-tartaric acid co-crystal Form B is characterized by an X-ray powder diffraction (XRPD) pattern comprising peaks as shown in Table 4, wherein the XRPD is made using CuK_{α1} radiation. In some embodiments, the L-tartaric acid co-crystal Form 1 is characterized by an X-ray powder diffraction (XRPD) pattern substantially as shown in FIG. 9, wherein the XRPD is made using CuK_{α1} radiation.

### Tartaric Acid Form 2

In some embodiments, the crystalline form of the compound of Formula (I) is L-tartaric acid co-crystal Form 2. In some embodiments, the crystalline form is a co-crystal having about 1:1 ratio of the compound of Formula (I) and L-tartaric acid. In some embodiments, the crystalline form is a co-crystal having about 1:1:1 ratio of the compound of Formula (I), L-tartaric acid, and water.

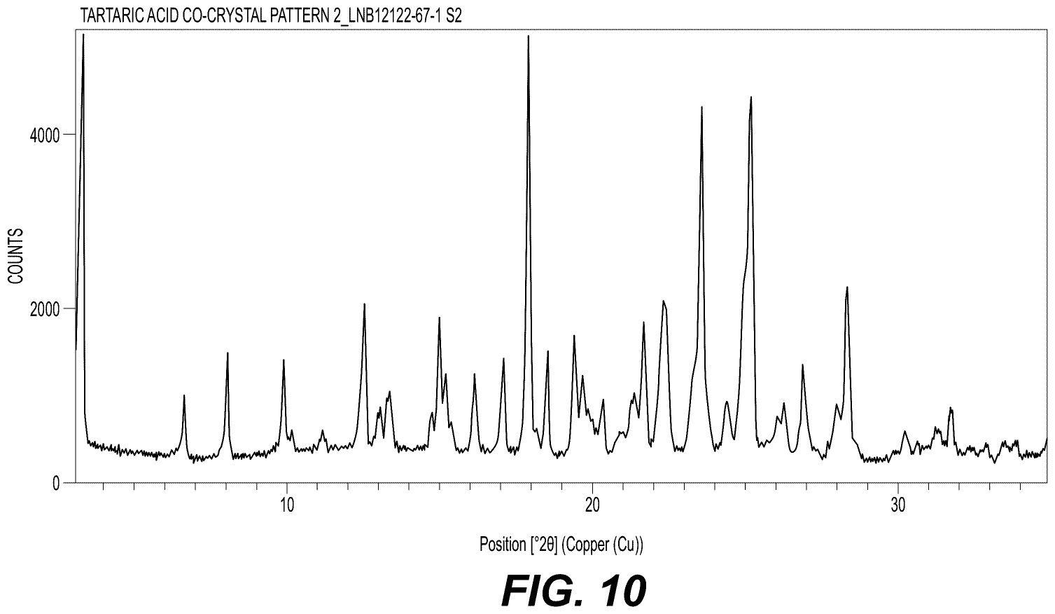

In some embodiments, the L-tartaric acid co-crystal Form 2 is characterized by an X-ray powder diffraction (XRPD) pattern comprising peaks at 3.3, 17.9, and 23.6 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 17.9, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 12.5, 17.9, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 12.5, 15.0, 17.9, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 12.5, 15.0, 17.9, 21.7, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 8.0, 9.9, 12.5, 15.0, 17.9, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks at 3.3, 8.0, 9.9, 12.5, 15.0, 17.9, 21.7, 23.6, and 25.1 degrees 2θ (±0.2 degrees 2θ), wherein the XRPD is made using CuK_{α1} radiation. In some embodiments, the L-tartaric acid co-crystal Form 2 is characterized by an X-ray powder diffraction (XRPD) pattern comprising peaks as shown in Table 5, wherein the XRPD is made using CuK_{α1} radiation. In some embodiments, the L-tartaric acid co-crystal Form 2 is characterized by an X-ray powder diffraction (XRPD) pattern substantially as shown in FIG. 10, wherein the XRPD is made using CuK_{α1} radiation.

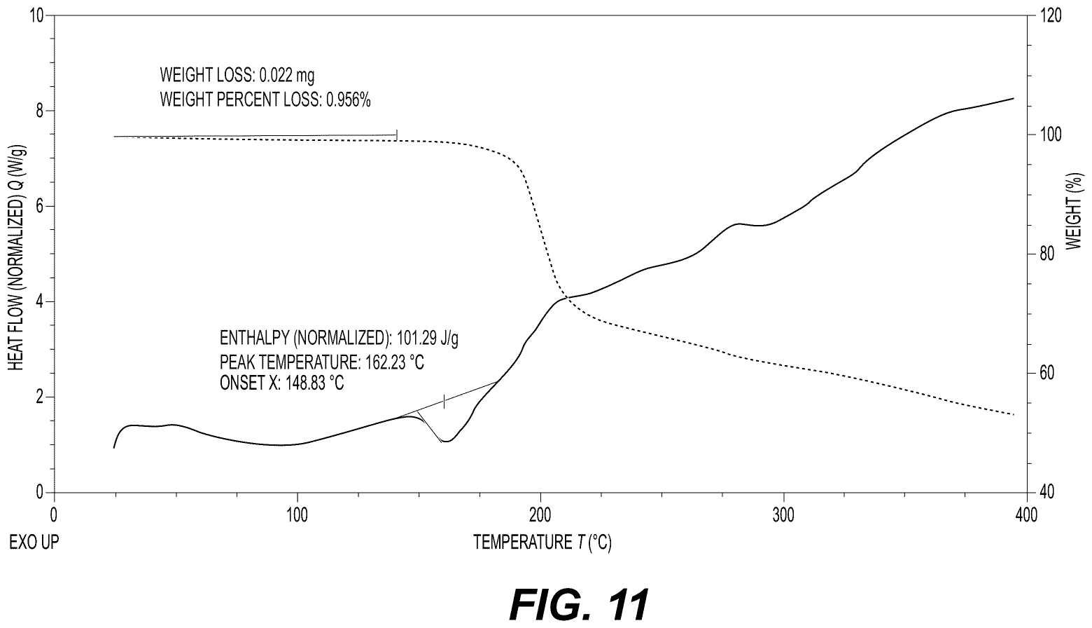

In some embodiments, the L-tartaric acid co-crystal Form 2 is characterized by a thermogravimetric analysis (TGA) graph substantially as shown in FIG. 11.

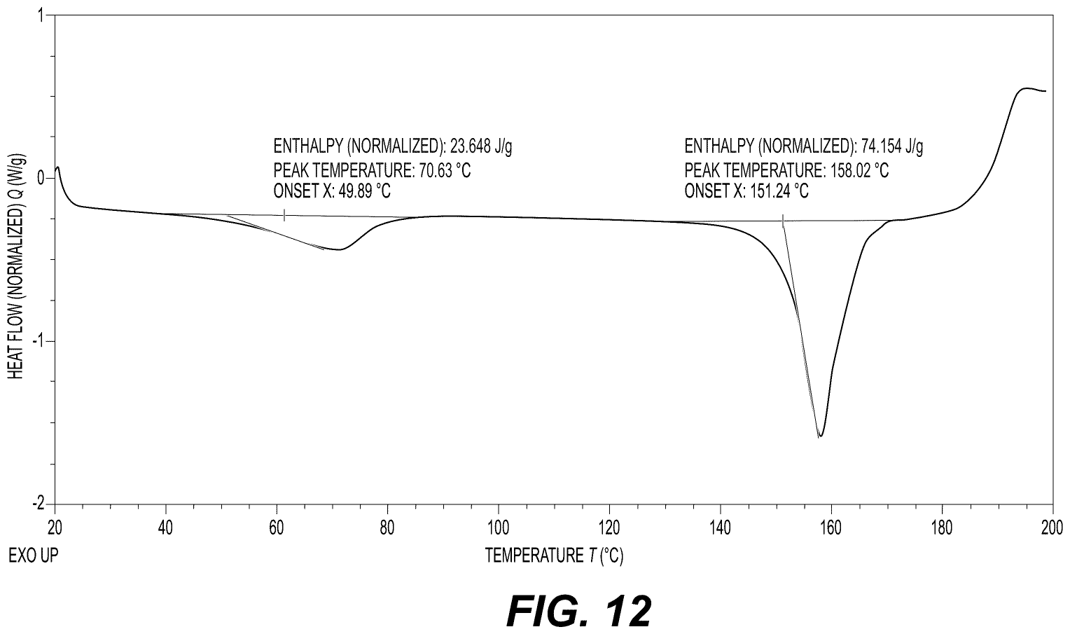

In some embodiments, the L-tartaric acid co-crystal Form 2 is characterized by a differential scanning calorimetry (DSC) graph having an endothermic peak at about 158° C. In some embodiments, the L-tartaric acid co-crystal Form 2 is characterized by a differential scanning calorimetry (DSC) graph substantially as shown in FIG. 12.

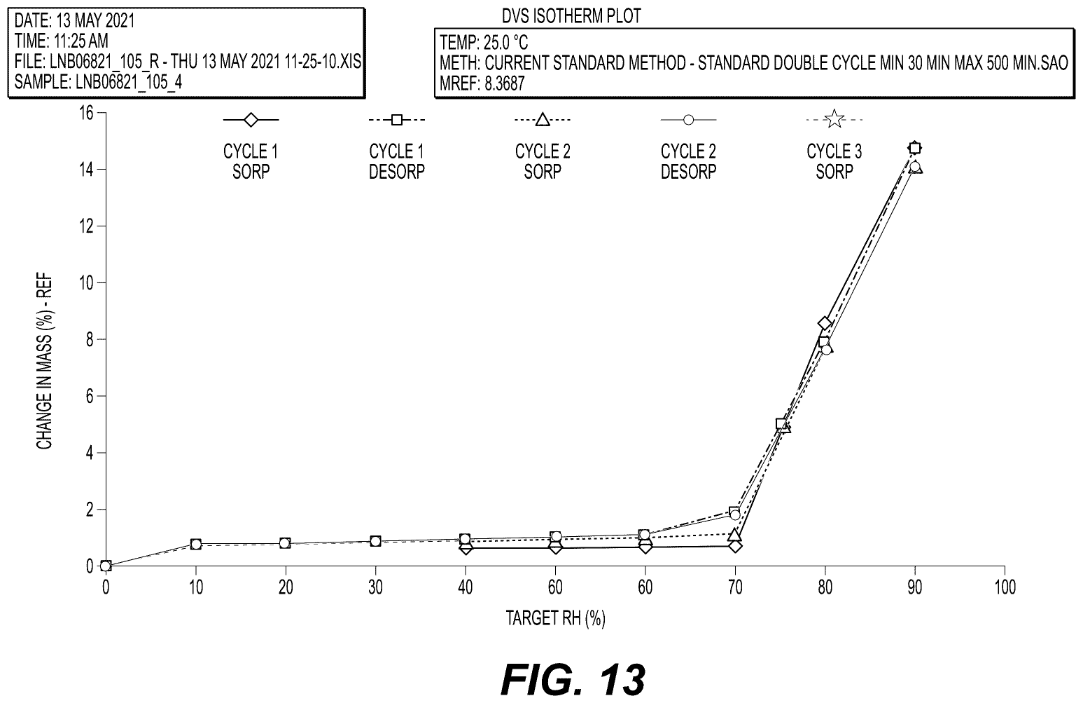

In some embodiments, the L-tartaric acid co-crystal Form 2 is characterized by a dynamic vapor sorption (DVS) plot substantially as shown in FIG. 13.

In some embodiments, L-tartaric acid co-crystal Form 2 of the compound of Formula (I) is stable under extended storage conditions, e.g., longer than about 1 month, about 3 months, about 6 months, about 1 year, about 2 years, about 3 years, or longer. In some embodiments, the extended storage conditions comprises storage at about 25° C. and about 60% relative humidity (RH). In some embodiments, the extended storage conditions comprises storage at about 40° C. and about 75% relative humidity (RH). In some embodiments, L-tartaric acid co-crystal Form 2 of the compound of Formula (I) has from about 95% to about 100% purity, e.g., greater than about 95%, about 96%, about 98%, about 99%, about 99.2%, about 99.5%, about 99.6%, about 99.7%, about 99.8%, about 99.9%, about 99.99% purity.

### Tartaric Acid Form 3

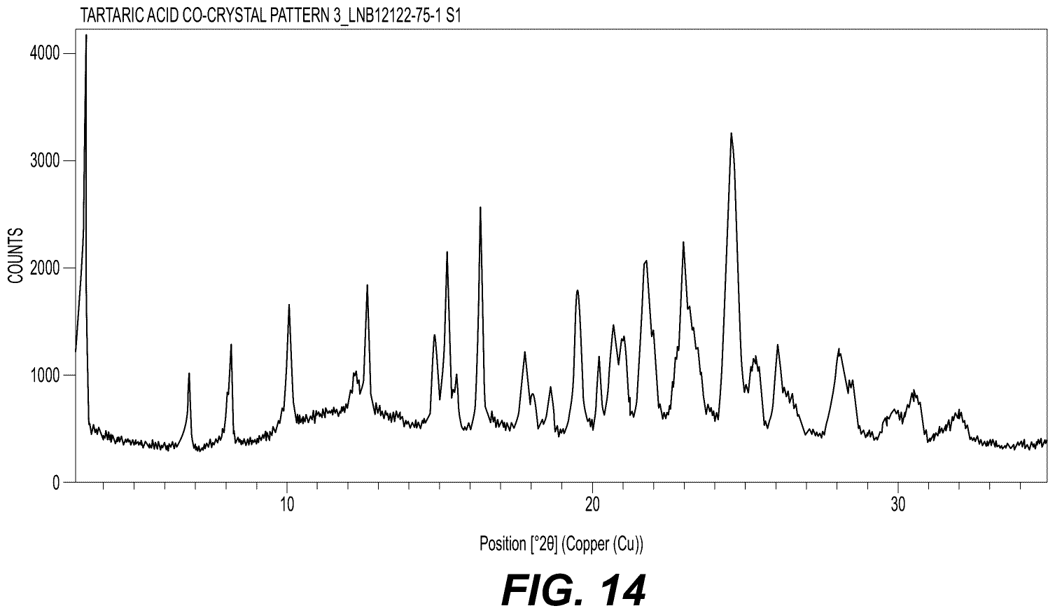

In some embodiments, the crystalline form is L-tartaric acid co-crystal Form 3. In some embodiments, the L-tartaric acid co-crystal Form 3 is characterized by an X-ray powder diffraction (XRPD) pattern comprising peaks at 3.4, 16.4, and 24.6 degrees 2θ (±0.2 degrees 2θ), wherein the XRPD is made using CuK_{α1} radiation. In some embodiments, the L-tartaric acid co-crystal Form 3 is characterized by an X-ray powder diffraction (XRPD) pattern comprising peaks at 3.4, 15.2, 16.4, and 24.6 degrees 2θ (±0.2 degrees 2θ), wherein the XRPD is made using CuK_{α1} radiation. In some embodiments, the L-tartaric acid co-crystal Form 3 is characterized by an X-ray powder diffraction (XRPD) pattern comprising peaks at 3.4, 15.2, 16.4, 23.0, and 24.6 degrees 2θ (±0.2 degrees 2θ), wherein the XRPD is made using CuK_{α1} radiation. In some embodiments, the L-tartaric acid co-crystal Form 3 is characterized by an X-ray powder diffraction (XRPD) pattern comprising peaks at 3.4, 12.6, 15.2, 16.4, 23.0, and 24.6 degrees 2θ (±0.2 degrees 2θ), wherein the XRPD is made using CuK_{α1} radiation. In some embodiments, the L-tartaric acid co-crystal Form 3 is characterized by an X-ray powder diffraction (XRPD) pattern comprising peaks at 3.4, 10.1, 12.6, 15.2, 16.4, 23.0, and 24.6 degrees 2θ (±0.2 degrees 2θ), wherein the XRPD is made using CuK_{α1} radiation. In some embodiments, the L-tartaric acid co-crystal Form 3 is characterized by an X-ray powder diffraction (XRPD) pattern comprising peaks as shown in Table 8, wherein the XRPD is made using CuK_{α1} radiation. In some embodiments, the L-tartaric acid co-crystal Form 3 is characterized by an X-ray powder diffraction (XRPD) pattern substantially as shown in FIG. 14, wherein the XRPD is made using CuK_{α1} radiation.

### Fumaric Acid Co-Crystal

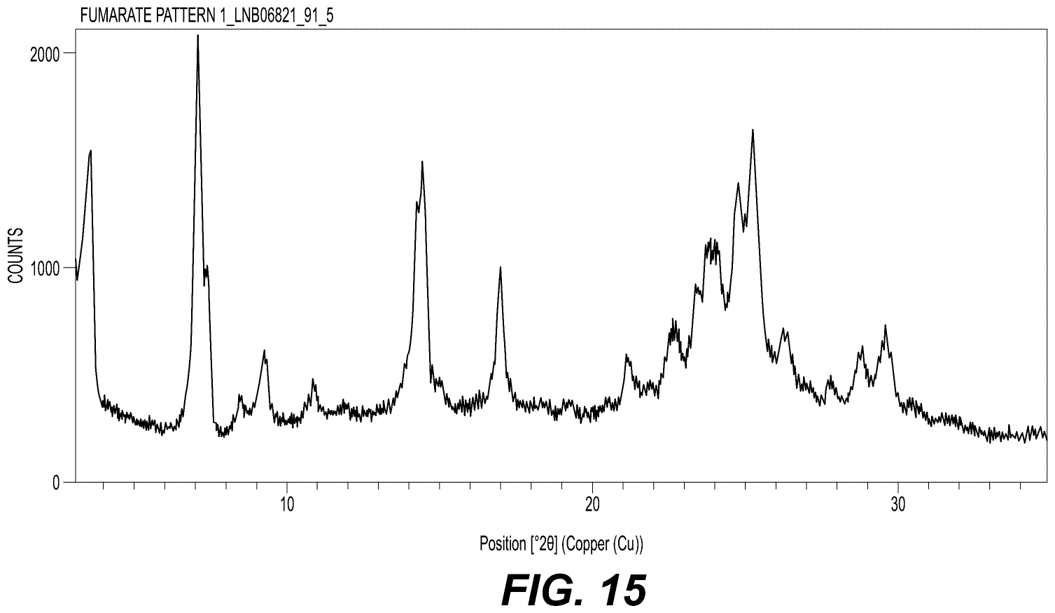

In some embodiments, the crystalline form is a fumaric acid co-crystal. In some embodiments, the fumaric acid co-crystal is characterized by an X-ray powder diffraction (XRPD) pattern comprising peaks at 14.5, 24.7, and 25.3 degrees 2θ (±0.2 degrees 2θ), wherein the XRPD is made using CuK_{α1} radiation. In some embodiments, the fumaric acid co-crystal is characterized by an X-ray powder diffraction (XRPD) pattern comprising peaks at 7.1, 14.5, 24.7, and 25.3 degrees 2θ (±0.2 degrees 2θ), wherein the XRPD is made using CuK_{α1} radiation. In some embodiments, the fumaric acid co-crystal is characterized by an X-ray powder diffraction (XRPD) pattern comprising peaks at 7.1, 14.2, 14.5, 24.7, and 25.3 degrees 2θ (±0.2 degrees 2θ), wherein the XRPD is made using CuK_{α1} radiation. In some embodiments, the fumaric acid co-crystal is characterized by an X-ray powder diffraction (XRPD) pattern comprising peaks at 3.5, 7.1, 14.2, 14.5, 24.7, and 25.3 degrees 2θ (±0.2 degrees 2θ), wherein the XRPD is made using CuK_{α1} radiation. In some embodiments, the fumaric acid co-crystal is characterized by an X-ray powder diffraction (XRPD) pattern comprising peaks at 3.5, 7.1, 14.2, 14.5, 23.9, 24.7, and 25.3 degrees 2θ (±0.2 degrees 2θ), wherein the XRPD is made using CuK_{α1} radiation. In some embodiments, the fumaric acid co-crystal is characterized by an X-ray powder diffraction (XRPD) pattern comprising peaks as shown in Table 10, wherein the XRPD is made using CuK_{α1} radiation. In some embodiments, the fumaric acid co-crystal is characterized by an X-ray powder diffraction (XRPD) pattern substantially as shown in FIG. 15, wherein the XRPD is made using CuK_{α1} radiation.

### Hydrochloride Salt

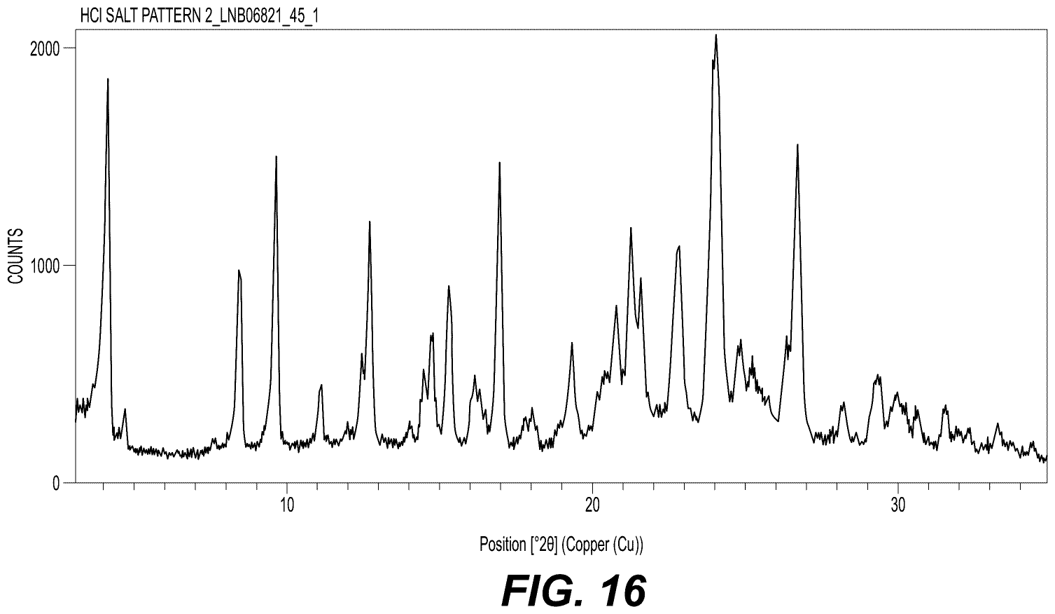

In some embodiments, the crystalline form is a hydrochloride. In some embodiments, the hydrochloride is characterized by an X-ray powder diffraction (XRPD) pattern comprising peaks at 12.7, 16.9, and 24.0 degrees 2θ (±0.2 degrees 2θ), wherein the XRPD is made using CuK_{α1} radiation. In some embodiments, the hydrochloride is characterized by an X-ray powder diffraction (XRPD) pattern comprising peaks at 12.7, 16.9, 21.2, and 24.0 degrees 2θ (±0.2 degrees 2θ), wherein the XRPD is made using CuK_{α1} radiation. In some embodiments, the hydrochloride is characterized by an X-ray powder diffraction (XRPD) pattern comprising peaks at 12.7, 16.9, 21.2, 22.8, and 24.0 degrees 2θ (±0.2 degrees 2θ), wherein the XRPD is made using CuK_{α1} radiation. In some embodiments, the hydrochloride is characterized by an X-ray powder diffraction (XRPD) pattern comprising peaks at 4.1, 12.7, 16.9, 21.2, 22.8, and 24.0 degrees 2θ (±0.2 degrees 2θ), wherein the XRPD is made using CuK_{α1} radiation. In some embodiments, the hydrochloride is characterized by an X-ray powder diffraction (XRPD) pattern comprising peaks at 4.1, 12.7, 16.9, 21.2, 22.8, 24.0, and 26.7 degrees 2θ (±0.2 degrees 2θ), wherein the XRPD is made using CuK_{α1} radiation. In some embodiments, the hydrochloride is characterized by an X-ray powder diffraction (XRPD) pattern comprising peaks as shown in Table 11, wherein the XRPD is made using CuK_{α1} radiation. In some embodiments, the hydrochloride is characterized by an X-ray powder diffraction (XRPD) pattern substantially as shown in FIG. 16, wherein the XRPD is made using CuK_{α1} radiation.

### Methanesulfonate Salt

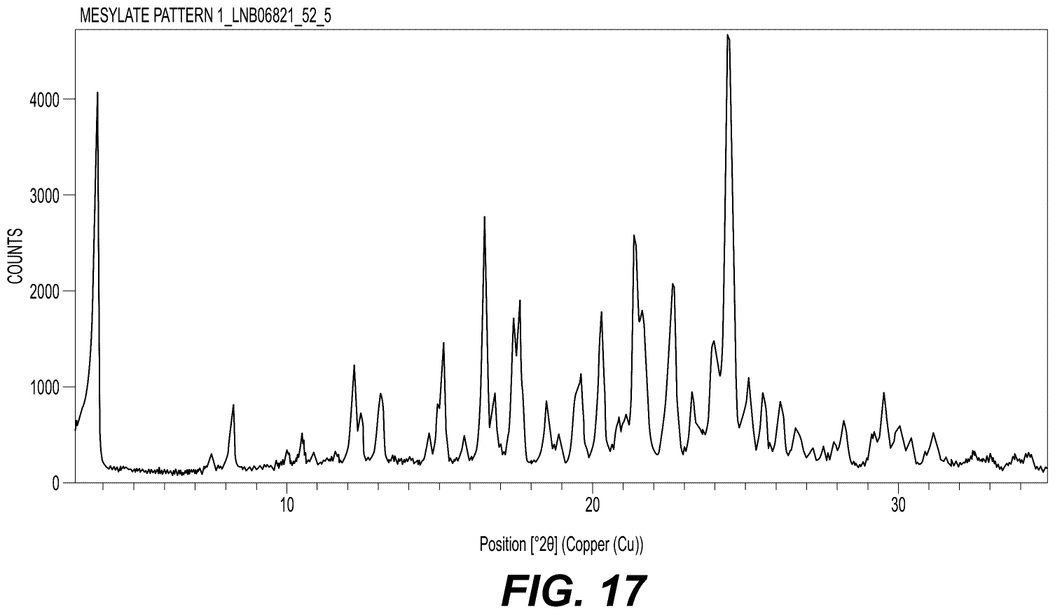

In some embodiments, the crystalline form is a methanesulfonate. In some embodiments, the methanesulfonate is characterized by an X-ray powder diffraction (XRPD) pattern comprising peaks at 16.5, 21.4, and 24.4 degrees 2θ (±0.2 degrees 2θ), wherein the XRPD is made using CuK_{α1} radiation. In some embodiments, the methanesulfonate is characterized by an X-ray powder diffraction (XRPD) pattern comprising peaks at 3.7, 16.5, 21.4, and 24.4 degrees 2θ (±0.2 degrees 2θ), wherein the XRPD is made using CuK_{α1} radiation. In some embodiments, the methanesulfonate is characterized by an X-ray powder diffraction (XRPD) pattern comprising peaks at 3.7, 16.5, 17.6, 21.4, and 24.4 degrees 2θ (±0.2 degrees 2θ), wherein the XRPD is made using CuK_{α1} radiation. In some embodiments, the methanesulfonate is characterized by an X-ray powder diffraction (XRPD) pattern comprising peaks at 3.7, 16.5, 17.6, 21.4, 21.6, and 24.4 degrees 2θ (±0.2 degrees 2θ), wherein the XRPD is made using CuK_{α1} radiation. In some embodiments, the methanesulfonate is characterized by an X-ray powder diffraction (XRPD) pattern comprising peaks at 3.7, 16.5, 17.6, 20.3, 21.4, 21.6, and 24.4 degrees 2θ (±0.2 degrees 2θ), wherein the XRPD is made using CuK_{α1} radiation. In some embodiments, the methanesulfonate is characterized by an X-ray powder diffraction (XRPD) pattern comprising peaks as shown in Table 12, wherein the XRPD is made using CuK_{α1} radiation. In some embodiments, the methanesulfonate is characterized by an X-ray powder diffraction (XRPD) pattern substantially as shown in FIG. 17, wherein the XRPD is made using CuK_{α1} radiation.

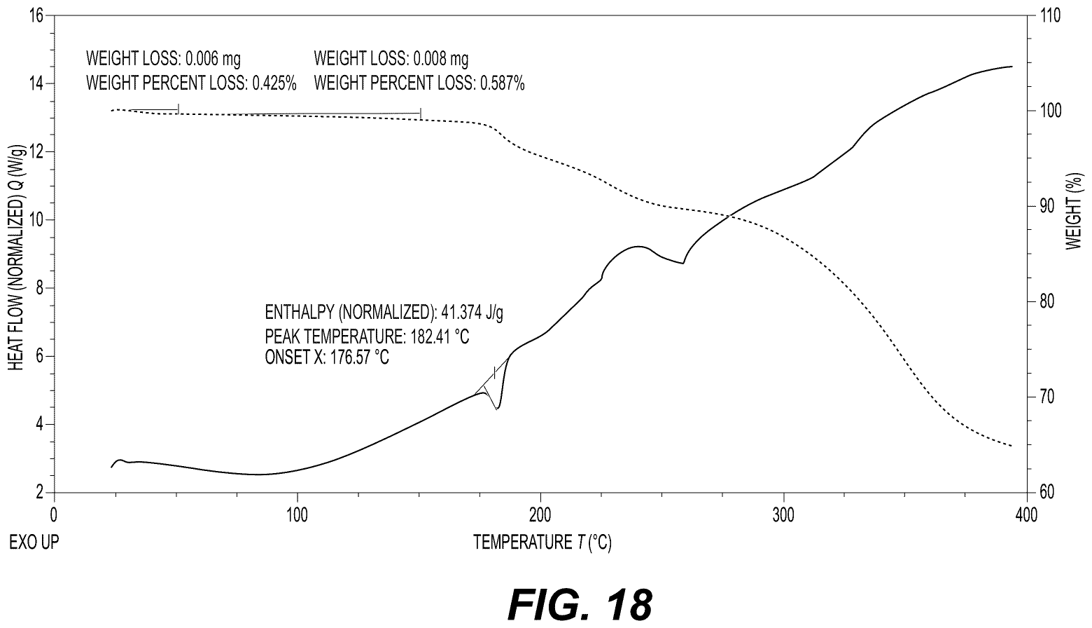

In some embodiments, the methanesulfonate is characterized by a thermogravimetric analysis (TGA) graph substantially as shown in FIG. 18.

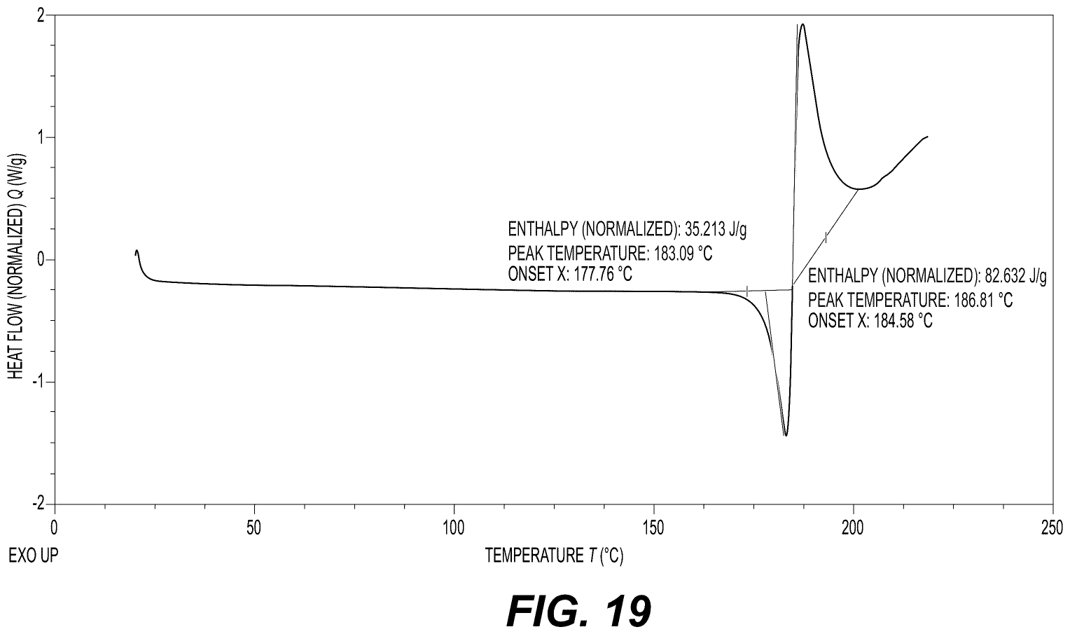

In some embodiments, the methanesulfonate is characterized by a differential scanning calorimetry (DSC) graph substantially as shown in FIG. 19.

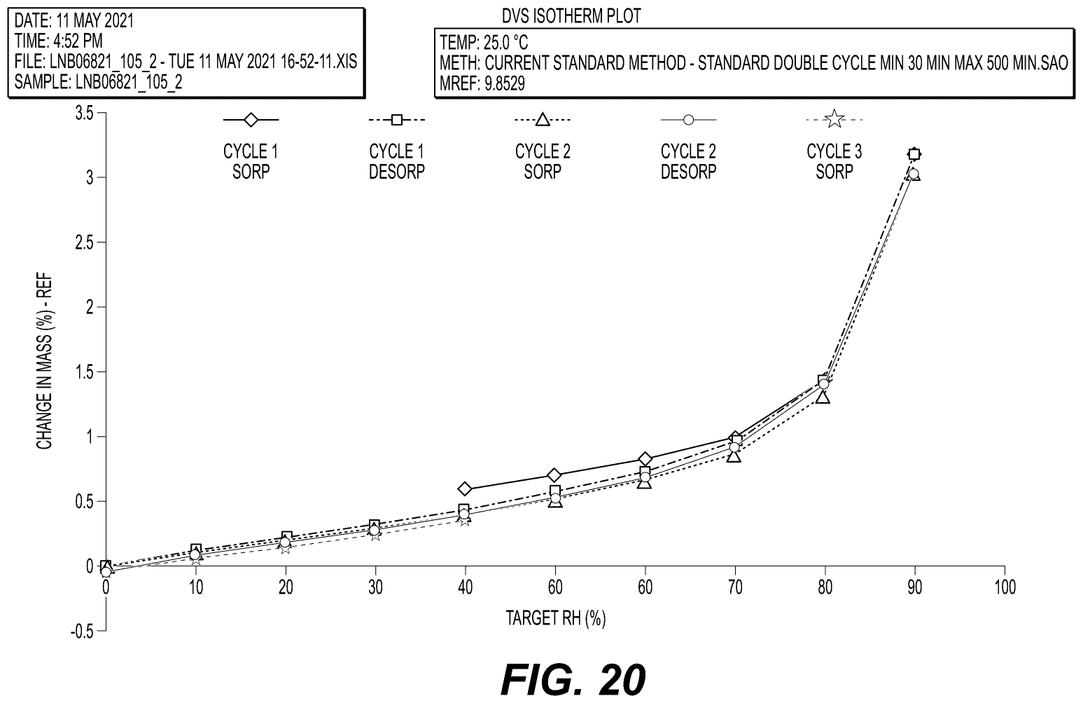

In some embodiments, the methanesulfonate is characterized by a dynamic vapor sorption (DVS) plot substantially as shown in FIG. 20.

### Phosphoric Acid Co-Crystal

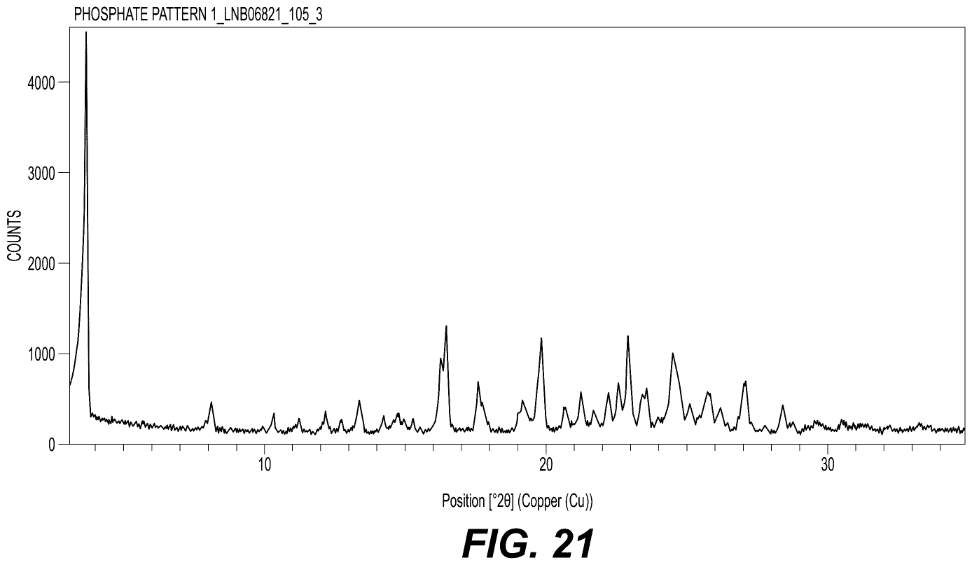

In some embodiments, the crystalline form is a phosphoric acid co-crystal. In some embodiments, the phosphoric acid co-crystal is characterized by an X-ray powder diffraction (XRPD) pattern comprising peaks at 3.7, 16.5, and 22.9 degrees 2θ (±0.2 degrees 2θ), wherein the XRPD is made using CuK_{α1} radiation. In some embodiments, the phosphoric acid co-crystal is characterized by an X-ray powder diffraction (XRPD) pattern comprising peaks at 3.7, 16.3, 16.5, and 22.9 degrees 2θ (±0.2 degrees 2θ), wherein the XRPD is made using CuK_{α1} radiation. In some embodiments, the phosphoric acid co-crystal is characterized by an X-ray powder diffraction (XRPD) pattern comprising peaks at 3.7, 16.3, 16.5, 22.9, and 24.5 degrees 2θ (±0.2 degrees 2θ), wherein the XRPD is made using CuK_{α1} radiation. In some embodiments, the phosphoric acid co-crystal is characterized by an X-ray powder diffraction (XRPD) pattern comprising peaks at 3.7, 16.3, 16.5, 17.6, 22.9, and 24.5 degrees 2θ (±0.2 degrees 2θ), wherein the XRPD is made using CuK_{α1} radiation. In some embodiments, the phosphoric acid co-crystal is characterized by an X-ray powder diffraction (XRPD) pattern comprising peaks at 3.7, 16.3, 16.5, 17.6, 19.8, 22.9, and 24.5 degrees 2θ (±0.2 degrees 2θ), wherein the XRPD is made using CuK_{α1} radiation. In some embodiments, the phosphoric acid co-crystal is characterized by an X-ray powder diffraction (XRPD) pattern comprising peaks as shown in Table 13, wherein the XRPD is made using CuK_{α1} radiation. In some embodiments, the phosphoric acid co-crystal is characterized by an X-ray powder diffraction (XRPD) pattern substantially as shown in FIG. 21, wherein the XRPD is made using CuK_{α1} radiation.

### IV. Compositions

The disclosure provides for, inter alia, compositions of one or more crystalline forms as disclosed herein. The compositions of the one or more crystalline forms can decrease the level of CaMKII in a cell.

In some embodiments, the composition comprises a crystalline form of the present disclosure, or a salt thereof. In some embodiments, the composition further comprises a carrier or excipient.

The crystalline form can be administered by any useful route and means, such as by oral or parenteral (e.g., intravenous) administration. Therapeutically effective amounts of the crystalline form may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

### A. Formulation

In some embodiments, the present disclosure provides a pharmaceutical composition, or pharmaceutical formulation, comprising a pharmaceutically effective amount of a crystalline form of the present disclosure, or a pharmaceutically acceptable salt thereof, and a pharmaceutically acceptable carrier or excipient. The pharmaceutical composition is capable of delivering an amount of a crystalline form of the disclosure sufficient to produce a therapeutically effective treatment as described further below. Also provided herein is a pharmaceutical formulation comprising a pharmaceutically effective amount of a crystalline form of Formula (I) or a pharmaceutically acceptable salt thereof, and a pharmaceutically acceptable carrier or excipient.

For preparing pharmaceutical compositions from the crystalline form or pharmaceutically acceptable salt of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material.

The crystalline forms herein are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Remington: The Science and Practice of Pharmacy, 23th edition, Adejare, A., ed. Academic Press: London, 2020. Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, comprise at least one active ingredient, as above defined, together with one or more acceptable carriers and optionally other therapeutic ingredients, particularly those additional therapeutic ingredients as discussed herein. The carrier(s) must be acceptable in the sense of being compatible with the other ingredients of the formulation and physiologically innocuous to the recipient thereof.

The formulations include those suitable for the administration routes described below. The formulations may conveniently be presented in unit dosage form and may be prepared by any of the methods well known in the art of pharmacy. Techniques and formulations generally are found in Remington: The Science and Practice of Pharmacy, 23th edition, Adejare, A., ed. Academic Press: London, 2020. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the crystalline form of the present invention.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Liquid form preparations include solutions, suspensions, and emulsions, for example, water or water/propylene glycol solutions. For parenteral injection, liquid preparations can be formulated in solution in aqueous polyethylene glycol solution.

Pharmaceutical formulations herein comprise a combination together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, solutions, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphoric acid; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations suitable for topical administration to the eye also include eye drops wherein the active ingredient is dissolved or suspended in a suitable carrier, especially an aqueous solvent for the active ingredient. The active ingredient can be present in such formulations in a concentration of about 0.5 to about 20%, such as about 0.5 to about 10%, for example about 1.5% w/w.

Aqueous solutions suitable for oral use can be prepared by dissolving the crystalline form or pharmaceutically acceptable salt of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolality.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the crystalline form or pharmaceutically acceptable salt of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration into a body cavity. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV, intratumoral, or intravitreal administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

### B. Administration

The crystalline form or pharmaceutically acceptable salt and compositions of the present invention can be delivered by any suitable means, including oral, parenteral and topical methods.

A crystalline form or composition of the present disclosure may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a crystalline form or composition of the present disclosure may be adjusted over the course of the treatment, based on the judgment of the administering physician.

The crystalline form or composition may be administered to an individual (e.g., a human) in an effective amount. In some embodiments, the crystalline form is administered once daily.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules.

The crystalline forms and compositions of the present invention can be co-administered with other agents. Co-administration includes administering the crystalline form or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the crystalline forms and compositions of the present invention can each be administered once a day, or two, three, or more times per day so as to provide the preferred dosage level per day.

Co-administration as used herein can refer to administration of unit dosages of the crystalline forms disclosed herein before, simultaneously, or after administration of unit dosages of one or more additional therapeutic agents, for example, administration of the crystalline form disclosed herein within seconds, minutes, or hours of the administration of one or more additional therapeutic agents. For example, in some embodiments, a unit dose of a crystalline form of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a crystalline form of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a crystalline form disclosed herein with one or more additional therapeutic agents generally refers to simultaneous or sequential administration of a crystalline form disclosed herein and one or more additional therapeutic agents, such that therapeutically effective amounts of each agent are present in the body of the patient.

In some embodiments, co-administration can be accomplished by co-formulation, i.e., preparing a single pharmaceutical composition including the compounds and compositions of the present invention and any other agent. Alternatively, the various components can be formulated separately.

The crystalline forms and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The crystalline forms described herein can be used in combination with one another, with other active agents known to be useful in modulating CaMKII, or with adjunctive agents that may not be effective alone, but may contribute to the efficacy of the active agent.

### V. Methods and/or Uses

### Methods of Preparation

In some embodiments, a method of the present disclosure is a method of preparing Form A of the compound of Formula (I), comprising: (a) warming a mixture of the compound of Formula (I) and a solvent comprising tert-butanol, t-BME, DMSO, DMA, DMF, NMP, THF, or trifluoroethanol, or a combination thereof, and (b) cooling the mixture, thereby preparing the Form A of the compound of Formula (I).

In some embodiments, a method of the present disclosure is a method of preparing Form B of the compound of Formula (I), comprising: (a) warming a mixture of the compound of Formula (I) and a solvent comprising 1,4-dioxane, 1-butanol, 1-propanol, 2-Me THF, 2-propanol, ethanol, ethyl acetate, isopropyl acetate, methanol, methylisobutyl ketone, or trifluorotoluene, or a combination thereof, and (b) cooling the mixture, thereby preparing the Form B of the compound of Formula (I).

In some embodiments, a method of the present disclosure is a method of preparing Form C of the compound of Formula (I), comprising: (a) warming a mixture of the compound of Formula (I) and a solvent comprising acetone, water, dichloromethane, or methyl ethyl ketone, or a combination thereof, and (b) cooling the mixture, thereby preparing the Form C of the compound of Formula (I).

Medical Methods and/or Uses

In some embodiments, a method of preventing or treating a CaMKII associated disease or condition described herein comprises administering a therapeutically effective amount of a crystalline form of the disclosure.

In some embodiments, a use of a therapeutically effective amount of a crystalline form of the disclosure is for preparation of a medicament in a method of preventing or treating a CaMKII associated disease or condition described herein.

In some embodiments, a therapeutically effective amount of a crystalline form of the disclosure is for use in a method of preventing or treating a CaMKII associated disease or condition described herein.

Since the crystalline form of the present invention has CaMKII inhibitory action, it is expected to be useful for the prophylaxis or treatment of, for example, cardiac diseases (cardiac hypertrophy, acute heart failure and chronic heart failure including congestive heart failure, cardiomyopathy, angina, myocarditis, atrial/ventricular arrhythmia, tachycardia, myocardial infarction, etc.), myocardial ischemia, venous insufficiency, post-myocardial infarction transition to heart failure, hypertension, cor pulmonale, arteriosclerosis including atherosclerosis (aneurysm, coronary arterial sclerosis, cerebral arterial sclerosis, peripheral arterial sclerosis, etc.), vascular thickening, vascular thickening/occlusion and organ damages after intervention (percutaneous coronary angioplasty, stent placement, coronary angioscopy, intravascular ultrasound, coronary thrombolytic therapy, etc.), vascular reocclusion/restenosis after bypass surgery, cardiac hypofunction after artificial heart lung surgery, respiratory diseases (cold syndrome, pneumonia, asthma, pulmonary hypertension, pulmonary thrombus/pulmonary embolism, etc.), bone disorders (nonmetabolic bone disorders such as bone fracture, refracture, bone malformation/spondylosis deformans, osteosarcoma, myeloma, dysostosis and scoliosis, bone defect, osteoporosis, osteomalacia, rickets, osteitis fibrosis, renal osteodystrophy, Paget's disease of bone, myelitis with rigidity, chronic rheumatoid arthritis, gonarthrosis and articular tissue destruction in similar disorders thereof, etc.), inflammatory diseases (diabetic complication such as retinopathy, nephropathy, nerve damage, macroangiopathy etc.; arthritis such as chronic rheumatoid arthritis, osteoarthritis, rheumatoid myelitis, periostitis etc.; inflammation after surgery/trauma; reduction of swelling; pharyngitis; cystitis; pneumonia; atopic dermatitis; inflammatory enteric diseases such as Crohn's disease, ulcerative colitis etc.; meningitis; inflammatory eye diseases; inflammatory pulmonary diseases such as pneumonia, silicosis, pulmonary sarcoidosis, pulmonary tuberculosis etc, and the like), allergic diseases (allergic rhinitis, conjunctivitis, gastrointestinal allergy, pollen allergy, anaphylaxis, etc.), drug dependence, neurodegenerative diseases (Alzheimer's disease, Parkinson's disease, amyotrophic lateral sclerosis, AIDS encephalopathy, etc.), central nervous system damage (disorders such as cerebral hemorrhage and cerebral infarction and aftereffects and complications thereof, head injury, spinal damage, cerebral edema, sensory dysfunction, sensory abnormality, autonomic dysfunction, abnormal autonomic function, multiple sclerosis etc.), dementia, disturbed memory, disturbed consciousness, amnesia, anxiety symptoms, nervous symptoms, unpleasant condition, mental disorders (depression, epilepsy, alcohol dependency, etc.), ischemic peripheral circulatory disorder, deep-vein thrombosis, occlusive peripheral circulatory disorder, arteriosclerosis obliterans (ASO), occlusive thromboangiitis, diabetes (type 1 diabetes, type 2 diabetes, pregnancy diabetes etc.), diabetic complications (nerve damage, nephropathy, retinopathy, cataract, macroangiopathy, osteopenia, diabetic hyperosmolar diabetic coma, infectious diseases, diabetic gangrene, xerostomia, deterioration in hearing, cerebrovascular damage, peripheral circulatory disorder, etc.), urinary incontinence, metabolic/nutritional disorders (obesity, hyperlipidemia, hypercholesterolemia, diabetes, impaired glucose tolerance, hyperuricemia, hyperkalemia, hypernatremia etc.), metabolic syndrome, vesceral obesity syndrome, male or female sexual dysfunction and the like, and for the prophylaxis or treatment of dysgeusia, smell disturbance, abnormal circadian rhythm of blood pressure, cerebrovascular damage (asymptomatic cerebrovascular damage, transient cerebral ischemia attack, stroke, cerebrovascular dementia, hypertensive encephalopathy, cerebral infarction, etc.), cerebral edema, cerebral circulatory disturbance, recurrence and aftereffects of cerebrovascular damages (neurological symptoms, mental symptoms, subjective symptoms, impairment of activities of daily living, etc.), kidney diseases (nephritis, glomerulonephritis, glomerulosclerosis, renal failure, thrombotic microangiopathy, diabetic nephropathy, nephrotic syndrome, hypertensive nephrosclerosis, complications of dialysis, organ damage including nephropathy by irradiation, etc.), erythrocytosis/hypertension/organ damage/vascular thickening after transplantation, rejection after transplantation, ocular disorders (glaucoma, ocular hypertension, etc.), thrombosis, multiple organ failure, endothelial dysfunction, hypertensive tinnitus, other circulatory diseases (ischemic cerebral circulatory disturbance, Raynaud's disease, Buerger's disease, etc.), chronic occlusive pulmonary diseases, interstitial pneumonia, carinii pneumonia, connective tissue disorders (e.g., systemic erythematosus, scleroderma, polyarteritis, etc.), liver disorders (hepatitis and cirrhosis including chronic types, etc.), portal hypertension, digestive disorders (gastritis, gastric ulcer, gastric cancer, disorder after gastric surgery, poor digestion, esophageal ulcer, pancreatitis, colon polyp, cholelithiasis, hemorrhoidal problem, esophageal and gastric variceal rupture, etc.), hematological/hematopoietic disorders (erythrocytosis, vascular purpura, autoimmune hemolytic anemia, disseminated intravascular coagulation syndrome, multiple myelosis, etc.), solid tumor, tumors (malignant melanoma, malignant lymphoma, digestive organs (e.g., stomach, intestine, etc.) cancers, etc.), cancers and cachexia associated therewith, cancer metastases, endocrine disorders (Addison's disease, Cushing's syndrome, pheochromocytoma, primary aldosteronism, etc.), Creutzfeldt-Jakob disease, urological/male genital diseases (cystitis, prostatic enlargement, prostate cancer, sexually transmitted diseases, etc.), gynecological disorders (menopausal disorders, pregnancy toxemia, endometriosis, uterine fibroid, ovarian diseases, mammary gland diseases, sexually transmitted diseases, etc.), diseases caused by environmental/occupational factor (e.g., radiation damage, damage from ultraviolet/infrared/laser beam, altitude sickness etc.), infectious diseases (viral infectious diseases of, for example, cytomegalovirus, influenza virus and herpesvirus, rickettsial infectious diseases, bacterial infectious diseases, etc.), toxemia (septicemia, septic shock, endotoxic shock, gram-negative septicemia, toxin shock syndrome, etc.), ear nose throat diseases (Ménière's disease, tinnitus, dysgeusia, vertigo, balance disorder, deglutition disorder etc.), cutaneous diseases (keloid, hemangioma, psoriasis, etc.), dialysis hypotension, myasthenia gravis, systemic diseases such as chronic fatigue syndrome, and the like, particularly cardiac diseases (particularly catecholaminergic polymorphic ventricular tachycardia, postoperative atrial fibrillation, heart failure, fatal arrhythmia) and the like, in a subject in need thereof.

Herein, the concept of prophylaxis of cardiac diseases include treatment of prognosis of myocardial infarction, angina attack, cardiac bypass surgery, thrombolytic therapy, coronary revascularization and the like, and the concept of treatment of cardiac diseases include suppress of progress or severity of heart failure (including both contractile failure HFrEF, and heart failure HFpEF with maintained ejection fraction), and maintenance of cardiac function when performing non-drug therapies (e.g., an implantable defibrillator, resection of cardiac sympathetic nerve, catheter ablation, cardiac pacemaker, intra aortic balloon pumping, auxiliary artificial heart, Batista operation, cell transplantation, gene therapy, heart transplantation and the like) for severe heart failure/arrhythmia, and the like. When the compound of the present invention is applied to prophylaxis or treatment of heart failure, improvement of heart contractility or atonicity is expected to be achieved by short-time administration, without side effects such as pressure decrease, tachycardia, reduced renal blood flow and the like, regardless of differences in causative diseases such as ischemic cardiac disease, cardiomyopathy, hypertension and the like and symptoms such as contractile failure, diastolic failure and the like. In some embodiments, long-term improvement of prognosis (survival rate, readmission rate, cardiac event rate etc.) can be achieved, in addition to short-term improvement of cardiac function. When the crystalline form of the present invention is applied to prophylaxis or treatment of arrhythmia, improvement or remission of the symptom is expected to be achieved, regardless of differences in etiology and atrial/ventricular. In addition, long-term improvement of prognosis (survival rate, readmission rate, cardiac event rate etc.) is expected to be achieved.

### VI. Examples

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

Many general references providing commonly known chemical synthetic schemes and conditions useful for synthesizing the disclosed compounds are available (see, e.g., Smith, March's Advanced Organic Chemistry: Reactions, Mechanisms, and Structure, 7^{th} edition, Wiley-Interscience, 2013.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. For example, disclosed compounds can be purified via silica gel chromatography. See, e.g., Introduction to Modem Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

Compounds were characterized using standard instrumentation methods.

Identification of the compound was carried out by hydrogen nuclear magnetic resonance spectrum (¹H-NMR) and mass spectrum (MS). ¹H-NMR was measured at 400 MHz, unless otherwise specified. In some cases, exchangeable hydrogen could not be clearly observed depending on the compound and measurement conditions. The designation br. or broad, used herein, refers to a broad signal. HPLC preparative chromatography was carried out by a commercially available ODS column in a gradient mode using water/methanol (containing formic acid) as eluents, unless otherwise specified.

Abbreviations as used herein have respective meanings as follows:

| | |
|---|---|
| Ac | acetate |
| ACN | acetonitrile |
| Bn | benzyl |
| br. s | broad singlet |
| Bu | butyl |
| DCM | dichloromethane |
| dd | doublet of doublets |
| ddd | doublet of doublet of doublets |
| DMF | dimethylformamide |
| DMSO | dimethylsulfoxide |
| DSC | differential scanning calorimetry |
| DVS | dynamic vapor sorption |
| ee | enantiomeric excess |
| equiv | equivalents |
| Et | ethyl |
| EtOAc | ethyl acetate |
| EtOH | ethanol |
| g | gram |
| GC | gas chromatography |
| h | hour |
| HPLC | high-pressure liquid chromatography |
| IPA | isopropyl alcohol |
| IPAc | isopropyl acetate |
| iPr | isopropyl |
| iPrOAc or IPAc | isopropyl acetate |
| kg | kilogram |
| L | liter |
| m | multiplet |
| M | molar |
| Me | methyl |
| MEK | methyl ethyl ketone |
| MeOH | methanol |
| 2-Me THF | 2-methyltetrahydrofuran |
| mg | milligram |
| MHz | megahertz |
| MIBK | methylisobutyl ketone |
| min | minute |
| mL | milliliter |
| mmol | millimole |
| mol | mole |
| MTBE or t-BME | methyl tert-butyl ether |
| N | normal |
| NMR | nuclear magnetic resonance |
| Ph | phenyl |
| PTFE | polytetrafluoroethylene |
| RH | relative humidity |
| s | singlet |
| t-Bu | tert-butyl |
| td | triplet of doublets |
| Tf | trifluoromethanesulfonate |
| TFE | trifluoroethanol |
| TGA | thermogravimetric analysis |
| THF | tetrahydrofuran |
| TMS | trimethylsilyl |
| vol | volume |
| wt | weight |
| XRPD | X-ray powder diffraction |
| δ | chemical shift |
| μL | microliter |

### General Procedures:

### X-ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Kapton or Mylar polymer film to support the sample. The multi-well plate was then placed into the diffractometer and analysed using Cu K radiation (α₁λ=1.54060 Å; α₂=1.54443 Å; β=1.39225 Å; α₁:α₂ ratio=0.5) running in transmission mode (step size 0.0130θ 2θ, step time 18.87 s) using 40 kV/40 mA generator settings. Data were visualized and images generated using the HighScore Plus 4.7 desktop application (PANalytical, 2017).

### Thermogravimetric Analysis/Differential Scanning Calorimetry (TGA/DSC)

Approximately, 5-10 mg of material was added into a pre-tared open aluminium pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Differential Scanning Calorimetry (DSC)

Approximately 1-5 mg of material was weighed into an aluminium DSC pan and sealed non-hermetically with an aluminium lid. The sample pan was then loaded into a TA Instruments Discovery DSC 2500 differential scanning calorimeter equipped with a RC90 cooler. The sample and reference were heated to 200° C., 210° C., 220° C. or 225° C. at a scan rate of 10° C./min unless otherwise indicated and the resulting heat flow response monitored.

### Dynamic Vapor Sorption (DVS)

Approximately 10-20 mg of sample was placed into a mesh vapor sorption balance pan and loaded into a DVS Intrinsic or Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 200 or 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined.

High-Performance Liquid Chromatography (HPLC) with ultraviolet (UV) detection
Instrument: Dionex Ultimate 3000
Column: YMC Pro Pack RS C-18 150 mm×4.6 mm, 3.0 μm
Column Temperature: 40° C.
Autosampler Temperature: Ambient
UV Wavelength: 230 nm
Injection Volume: 10 μL
Flow Rate: 1.0 mL/min
Mobile Phase A: 50 mM ammonium acetate in water
Mobile Phase B: Acetonitrile

### Gradient Program:

| Time (minutes) | Solvent B [%] |
|---|---|
| 0.0 | 35 |
| 10.0 | 35 |
| 18.0 | 90 |
| 23.0 | 90 |
| 23.1 | 35 |
| 30.0 | 35 |

### Mass Spectrometry (MS)

Instrument: Agilent 6410 QqQ using Agilent Infinity
Column: X-bridge C18, 50 mm×3 mm, 3.5 μm
Column Temperature: 40° C.
Autosampler Temperature: Ambient
Detection Parameters: UV 210 nm monitor only
Mass range 100 to 1500 m/z
MS+ESI Fragmentor 135 V
Capillary 300° C.
Injection Volume: 1 μL
Flow Rate: 1.0 mL/min
Mobile Phase A: 0.1% v/v formic acid in water
Mobile Phase B: 0.1% v/v formic acid in acetonitrile
Diluent: Methanol
Working Concentration: 0.1 mg/mL

### Gradient Program:

| Time (minutes) | Solvent B [%] |
|---|---|
| 0.0 | 5 |
| 8.0 | 95 |
| 10.0 | 95 |
| 10.1 | 5 |
| 14.0 | 5 |

### Example 1. Preparation of Crystalline Forms of Formula (I)

Amorphous Formula (I) was prepared by dissolving 330 mg Formula (I) compound in 1,4-dioxane (33 mL) in a glass container. This mixture was heated to about 40° C. to aid dissolution, then divided into aliquots of about 10 mg each in vials. The vials were frozen at −18° C., and lyophilized for about 15 hours. The resulting solid was confirmed to be amorphous by XRPD.

To about 10 mg amorphous Formula (I), a solvent was added in aliquots of 100 μL, and then heated on a hot plate to about 40° C. to aid dissolution. Solvent addition was continued until Formula (I) had completely dissolved or 2 mL of the appropriate solvent system had been added (<5 mg/mL). After the assessment was completed, the clear solutions were uncapped and allowed to evaporate at ambient temperature (ca. 20° C.) to recover solids. The slurries were isolated via centrifuge filtration (nylon, 0.22 μm). The isolated solids were analyzed by XRPD to determine the form. The following solvent systems were tested:

| Number | Solvent System (% v/v) |
|---|---|
| 1 | 1,4-Dioxane |
| 2 | 1-Butanol |
| 3 | 1-Propanol |
| 4 | 2-Methyl THF |
| 5 | 2-Propanol |
| 6 | Acetic acid |
| 7 | Acetone |
| 8 | Acetone:Water 95:5 |
| 9 | Acetone:Water 80:20 |
| 10 | Acetonitrile |
| 11 | Acetonitrile:Water 90:10 |
| 12 | Anisole |
| 13 | Dichloromethane |
| 14 | Diisopropyl ether |
| 15 | Dimethylsulfoxide |
| 16 | Ethanol |
| 17 | Ethyl acetate |
| 18 | Ethylene glycol |
| 19 | Heptane |
| 20 | Isopropyl acetate |
| 21 | Methanol |
| 22 | Methylethyl ketone |
| 23 | Methylisobutyl ketone |
| 24 | N,N-Dimethylacetamide |
| 25 | N,N-Dimethylformamide |
| 26 | N-Methylpyrrolidone |
| 27 | Propylene carbonate |
| 28 | tert-Butanol |
| 29 | tert-Butylmethyl ether |
| 30 | Tetrahydrofuran |
| 31 | Toluene |
| 32 | Water |

A primary polymorph screen was carried out in 26 solvent systems in order to identify novel polymorphic or solvated forms of Formula (I). The solvent systems were selected based on the results of the approximate solubility screen. Crystallization conditions investigated included thermal cycling, cooling, anti-solvent addition, evaporation, solvent drop grinding and vapor diffusion. The polymorph screen was carried out as follows:

About 2 g of Formula (I) compound was dissolved in 66 mL of 1,4-dioxane. The clear, colourless solution was divided between 25×4 mL screw cap vials to give ca. 80 mg per vial. The experiments were frozen at −18° C., and transferred to a desiccator attached to a freeze dryer. The experiments were dried via lyophilization for ca. 72 hours. The lyophilized material was analyzed by XRPD.

To ca. 80 mg of amorphous material the appropriate solvent system was added to form slurries. See below for the volumes of solvent used.

| Solvent System (% v/v) | Volume Added (μL) |
|---|---|
| 1,4-Dioxane | 450 |
| 1-Butanol | 800 |
| 1-Propanol | 1200 |
| 2-Methyl THF | 900 |
| 2-Propanol | 1300 |
| Acetic acid | 350 |
| Acetone | 500 |
| Acetone:Water 95:5 | 450 |
| Acetonitrile:Water 90:10 | 450 |
| Dichloromethane | 350 |
| Dimethylsulfoxide | 350 |
| Ethanol | 1400 |
| Ethyl acetate | 1400 |
| Isopropyl acetate | 1400 |
| Methanol | 1700 |
| Methylethyl ketone | 1100 |
| Methylisobutyl ketone | 1700 |
| N,N-Dimethylacetamide | 350 |
| N,N-Dimethylformamide | 350 |
| N-Methylpyrrolidone | 350 |
| tert-Butanol | 2000 |
| tert-Butylmethyl ether | 3500 |
| Tetrahydrofuran | 500 |
| Toluene | 3000 |
| Trifluoroethanol | 350 |
| Trifluorotoluene | 500 |

Temperature cycling was carried out between ambient temperature (ca. 20° C.) and 40° C. for ca. 72 h with agitation. Hold periods of 4 h at ambient temperature (ca. 20° C.) and 4 h hold periods at 40° C. were carried out. After temperature cycling, the solids were isolated by centrifugation using 0.22 μm nylon filters and analyzed by XRPD. The saturated solutions obtained were then divided into five for further experiments—cooling, anti-solvent addition, evaporation, solvent drop grinding and vapor diffusion. For the solvent drop grinding experiments a further batch of amorphous material was prepared. 20 mL of 1,4-dioxane was added to ca. 543 mg of Formula (I) to dissolve. The solution was divided between 27×2 mL push cap vials to give ca. 20 mg per vial. The experiments were frozen and dried via lyophilization for ca. 21 h. The lyophilized material was analyzed by XRPD.

Subsequent crystallization experiments were carried out as follows:
Cooling: Saturated solutions were stored in the fridge (5° C.) for ca. 3 days then moved to −18° C. where no or an insufficient amount of solids for analysis was observed.
Evaporation: Saturated solutions were allowed to evaporate at ambient temperature (ca. 20° C.) and pressure to recover solid material. Acetic acid, DMA and NMP solutions were allowed to evaporate at 60° C.
Anti-solvent addition: Anti-solvent was added dropwise with stirring to saturated solutions at ambient temperature (ca. 20° C.). The experiments were stored at 5° C. to encourage precipitation.
Solvent drop grinding: 1 to 2 drops of saturated solution was added to 20 mg of amorphous material and shaken using a bead mill with 2×2.8 mm bead mill beads. The experiments were shaken at 4500 rpm in 10×90 second intervals with a 10 second pause between each interval. 4 cycles in total were carried out.
Vapor diffusion: 2 mL of anti-solvent was added to 20 mL vials and the 2 mL vial containing the saturated solution added. The 20 mL vials were capped and stored at ambient temperature (ca. 20° C.).
All solids were analyzed by XRPD in the first instance. Potential new forms were re-analyzed by XRPD after drying at 40° C. at ambient pressure for ca. 22 h. Where there was sufficient material was obtained, TG/DSC, NMR, FT-IR and PLM analysis was carried out.

### Form A

Solids of anhydrous free base Formula (I) Form A were isolated after temperature cycling in tert-butanol, and t-BME. Free base Form A was obtained from anti-solvent addition to solutions in DMSO, DMA, DMF, NMP, THF, and trifluoroethanol. Solids of Form A were also observed from solvent drop grinding in methylisobutyl ketone, DMA, and tert-butanol. Form A was also recovered from vapor diffusion experiments with a solution in DMSO.

TABLE 1
XRPD peak list for Formula (I) Form A

| Pos. [°2θ] | d-spacing [Å] | Height [cts] | Rel. Int. [%] |
|---|---|---|---|
| 4.7 | 18.97432 | 4510.13 | 100 |
| 9.4 | 9.44073 | 155.7 | 3.45 |
| 11.2 | 7.86657 | 197.9 | 4.39 |
| 12.0 | 7.36312 | 966.61 | 21.43 |
| 12.4 | 7.13578 | 453.85 | 10.06 |
| 13.1 | 6.77455 | 2815.31 | 62.42 |
| 13.4 | 6.62467 | 1735.91 | 38.49 |
| 14.0 | 6.30874 | 455.44 | 10.1 |
| 14.4 | 6.13886 | 1469.93 | 32.59 |
| 16.1 | 5.50832 | 4172.51 | 92.51 |
| 16.7 | 5.31569 | 910.86 | 20.2 |
| 17.2 | 5.14073 | 3468.81 | 76.91 |
| 18.3 | 4.84346 | 1411.34 | 31.29 |
| 19.4 | 4.58099 | 1814.16 | 40.22 |
| 19.8 | 4.47308 | 562.44 | 12.47 |
| 20.9 | 4.25445 | 1061.49 | 23.54 |
| 21.2 | 4.18218 | 554.82 | 12.3 |
| 22.1 | 4.01196 | 1808.51 | 40.1 |
| 22.7 | 3.92253 | 1244.31 | 27.59 |
| 23.5 | 3.78937 | 562.94 | 12.48 |
| 24.1 | 3.69001 | 1119.72 | 24.83 |
| 24.7 | 3.59887 | 1975.31 | 43.8 |
| 25.1 | 3.54125 | 1705.79 | 37.82 |
| 26.0 | 3.42132 | 735.76 | 16.31 |
| 26.5 | 3.36009 | 3526.5 | 78.19 |
| 27.0 | 3.30517 | 1547.46 | 34.31 |
| 28.2 | 3.15648 | 788.91 | 17.49 |
| 29.1 | 3.06439 | 723 | 16.03 |
| 29.4 | 3.03672 | 679.95 | 15.08 |
| 32.8 | 2.72624 | 225.7 | 5 |
| 33.7 | 2.65711 | 229.78 | 5.09 |
| 34.2 | 2.61668 | 59.46 | 1.32 |

### Form B

Solids of anhydrous free base Formula (I) Form B were obtained after temperature cycling in 1,4-dioxane, 1-butanol, 1-propanol, 2-Me THF, 2-propanol, ethanol, ethyl acetate, isopropyl acetate, methanol, methylisobutyl ketone, and trifluorotoluene. Form B was also recovered from solvent drop grinding experiments in 1-butanol, 1-propanol, 2-Me THF, 2-propanol, acetonitrile:water (90:10), DMSO, ethanol, ethyl acetate, isopropyl acetate, ane methanol solvent systems.

TABLE 2
XRPD peak list for Formula (I) Form B

| Pos. [°2θ] | d-spacing [Å] | Height [cts] | Rel. Int. [%] |
|---|---|---|---|
| 4.7 | 18.92138 | 993.4 | 49.21 |
| 12.0 | 7.39954 | 598.06 | 29.63 |
| 13.0 | 6.78118 | 145.86 | 7.23 |
| 13.9 | 6.34472 | 619.59 | 30.7 |
| 14.4 | 6.12881 | 814.32 | 40.34 |
| 15.2 | 5.82491 | 803.87 | 39.82 |
| 16.1 | 5.50599 | 2018.55 | 100 |
| 16.4 | 5.38841 | 976.43 | 48.37 |
| 16.9 | 5.23606 | 1235.75 | 61.22 |
| 17.7 | 5.00299 | 1110.07 | 54.99 |
| 18.8 | 4.71987 | 244.61 | 12.12 |
| 19.8 | 4.47394 | 270.12 | 13.38 |
| 20.1 | 4.41202 | 442.02 | 21.9 |
| 21.1 | 4.20339 | 229.24 | 11.36 |
| 21.6 | 4.1151 | 561.81 | 27.83 |
| 22.1 | 4.02089 | 517.34 | 25.63 |
| 22.3 | 3.97516 | 341.54 | 16.92 |
| 22.6 | 3.93523 | 467.62 | 23.17 |
| 23.2 | 3.83284 | 700.14 | 34.69 |
| 24.0 | 3.70188 | 537.2 | 26.61 |
| 24.7 | 3.59913 | 625.93 | 31.01 |
| 25.7 | 3.46271 | 651.14 | 32.26 |
| 26.6 | 3.34922 | 1288.38 | 63.83 |
| 26.9 | 3.31066 | 312.59 | 15.49 |
| 27.4 | 3.25033 | 211.47 | 10.48 |
| 28.2 | 3.16544 | 375.09 | 18.58 |
| 28.5 | 3.1269 | 456.16 | 22.6 |
| 29.3 | 3.04464 | 222.12 | 11 |
| 30.7 | 2.91035 | 56.61 | 2.8 |
| 31.7 | 2.81836 | 139.28 | 6.9 |
| 33.2 | 2.69323 | 133.34 | 6.61 |
| 33.8 | 2.65325 | 72.15 | 3.57 |

### Form C

Solids of anhydrous free base Formula (I) Form C were obtained after temperature cycling in acetone, acetone:water 95:5% v/v, DCM, and methyl ethyl ketone. Solids of Form C were also isolated from anti-solvent addition to a solution in ethyl acetate, and from solvent-drop grinding in 1,4-dioxane and THF.

6.2 mL of acetone was added to ca. 1 g of amorphous Formula (I) compound in a 20 mL screw cap vial to create a slurry. Temperature cycling was carried out between ambient temperature (ca. 20° C.) and 40° C., for ca. 4 days. Hold periods of 4 h at ambient temperature (ca. 20° C.) and 4 h hold periods at 40° C. were carried out. At the end of temperature cycling the slurry was isolated by Büchner filtration with Grade 1 filter paper. The solids were dried under vacuum at ambient temperature (ca. 20° C.) for ca. 21 h to give Formula (I) Form C (576 mg, 55%), which was then analyzed by XRPD, TGA/DSC, DSC, and DVS. HPLC purity analysis indicated 98.8% area purity. The input purity was 98.3% area.

An exemplary XRPD for Formula (I) Form C is shown in FIG. 4. The list of peaks are shown in Table 3 below.

TABLE 3
XRPD peak list for Formula (I) Form C

| Pos. [°2θ] | d-spacing [Å] | Height [cts] | Rel. Int. [%] |
|---|---|---|---|
| 7.8 | 11.30772 | 1533.29 | 18.59 |
| 8.1 | 10.92275 | 1180.27 | 14.31 |
| 8.6 | 10.31248 | 2668.9 | 32.36 |
| 9.7 | 9.109 | 2483.9 | 30.12 |
| 11.1 | 7.97235 | 124.8 | 1.51 |
| 12.2 | 7.25861 | 187.96 | 2.28 |
| 12.7 | 6.98744 | 2430.48 | 29.47 |
| 14.4 | 6.16683 | 8247.46 | 100 |
| 15.2 | 5.83486 | 1743.13 | 21.14 |
| 15.3 | 5.78879 | 2004.11 | 24.3 |
| 15.7 | 5.6459 | 1007.24 | 12.21 |
| 16.3 | 5.44767 | 3841.91 | 46.58 |
| 17.2 | 5.14301 | 667.34 | 8.09 |
| 18.0 | 4.93525 | 219.03 | 2.66 |
| 18.3 | 4.84972 | 871.15 | 10.56 |
| 19.5 | 4.55491 | 353.9 | 4.29 |
| 20.8 | 4.27086 | 1359.21 | 16.48 |
| 22.2 | 4.00035 | 7222.65 | 87.57 |
| 23.1 | 3.85227 | 1333.82 | 16.17 |
| 23.6 | 3.77111 | 1208.69 | 14.66 |
| 24.3 | 3.66011 | 3133.14 | 37.99 |
| 25.2 | 3.52525 | 4820.84 | 58.45 |
| 25.5 | 3.49517 | 1315.35 | 15.95 |
| 25.8 | 3.45159 | 698.28 | 8.47 |
| 26.1 | 3.41775 | 1970.69 | 23.89 |
| 26.4 | 3.37749 | 1829.58 | 22.18 |
| 27.0 | 3.30202 | 505.52 | 6.13 |
| 27.7 | 3.21509 | 3328.65 | 40.36 |
| 30.2 | 2.95988 | 115.25 | 1.4 |
| 30.6 | 2.91843 | 371.4 | 4.5 |
| 31.0 | 2.88113 | 284.97 | 3.46 |
| 31.7 | 2.82232 | 862.86 | 10.46 |
| 33.4 | 2.68179 | 494.4 | 5.99 |
| 34.4 | 2.60414 | 342.01 | 4.15 |

TGA showed a loss of 0.1% followed by a loss of 0.6% (FIG. 5). The weight losses corresponded to 0.2 equivalents of water or 0.06 equivalents of acetone. Simultaneous DSC analysis carried out at 10° C./min showed two endothermic events with onsets ca. 173° C. (peak at 178° C.) and 187 SC (peak at 189° C.

DSC analysis was also carried out with a ramping rate of 1° C./min (FIG. 6). The first heating cycle showed one endothermic event with onset ca. 181° C., and an endothermic peak at about 184° C.

DVS analysis showed Formula (I) Form C to be slightly hygroscopic with 0.3% uptake at 80% RH (FIG. 7). Post-DVS XRPD analysis showed Formula (I) Form C was retained.

### Example 2. Preparation of Crystalline Salt Forms and Co-crystals of Formula (I)

A salt screen was carried out using 24 acids to identify potential salts of Formula (I). The following counterions and solvent systems 2-propanol, acetone:water (95:5), acetonitrile:water (90:10), dichoromethane, ethyl acetate, and THF were selected for salt screening.

Hydrochloric acid
Naphthalene-1,5-disulfonic acid
Sulfuric acid
Ethane-1,2-disulfonic acid
Cyclamic acid
Ethanesulfonic acid
2-Hydroxyethanesulfonic acid
p-Toluenesulfonic acid
Methanesulfonic acid
Naphthalene-2-sulfonic acid
Benzenesulfonic acid
Oxalic acid
2,2-Dichloroacetic acid
Maleic acid
L-Aspartic acid
Phosphoric acid
(+)-Camphor-10-sulfonic acid
Glutamic acid
Ketoglutaric acid
Malonic acid
Gentisic acid
(+)-L-Tartaric acid
Fumaric acid
Citric acid

The salt screen was carried out as follows: to about 30 mg of Formula (I) Form B in 2 mL push cap vials, 300 μL of the appropriate solvent was added to dissolve or create a slurry. 1.05 equivalents of counterion were weighed into separate vials. A solution or slurry of the counterion in 200-300 μL of appropriate solvent was added to the free base. Where the counterion was a liquid, it was added to the free base slurry from a stock solution in the allocated solvent. For 2-hydroxyethanesulfonic acid, a stock solution was prepared in dichloromethane and used for miscibility in 2-propanol, ethyl acetate and THF. Similarly, a stock solution of 2-hydroxyethanesulfonic acid was prepared in 95:5% v/v acetone:water and used for the 90:10%/v acetonitrile:water experiment.

The experiments were temperature cycled between ambient temperature (ca. 20° C.) and 50° C. in 4 hour cycles for ca. 3 days. Hold periods of 4 h at ambient temperature (ca. 20° C.) and 4 h hold periods at 40° C. were carried out. After temperature cycling, all solids were isolated by centrifugation (nylon, 0.22 μm) and analyzed by XRPD. Potential salts were dried at 40° C. at atmospheric pressure for ca. 24 h and re-analyzed by XRPD and then stored at 40° C./75% RH for ca. 22 h and re-analyzed by XRPD.

### Formula (I) L-Tartaric Acid Co-crystals

### L-Tartaric Acid Form 1

L-Tartaric acid co-crystal Form 1 was isolated from the experiment in isopropanol. XRPD analysis of crystals after drying at 40° C. showed Form 1. The XRPD peak list is shown in Table 4 below.

TABLE 4
XRPD peak list for Formula (I) L-Tartaric Acid Co-crystal Form 1

| Pos. [°2θ] | d-spacing [Å] | Height [cts] | Rel. Int. [%] |
|---|---|---|---|
| 4.7 | 18.9821 | 117.5 | 25.67 |
| 16.0 | 5.51896 | 102.53 | 22.4 |
| 17.2 | 5.15596 | 81.25 | 17.75 |
| 20.7 | 4.29292 | 127.31 | 27.81 |
| 21.7 | 4.0954 | 109.56 | 23.93 |
| 23.2 | 3.83129 | 133.17 | 29.09 |
| 25.5 | 3.48998 | 310.75 | 67.89 |
| 26.1 | 3.41779 | 215.78 | 47.14 |
| 26.9 | 3.30686 | 132.69 | 28.99 |
| 28.1 | 3.17389 | 457.76 | 100 |
| 28.2 | 3.15726 | 338.79 | 74.01 |
| 29.1 | 3.0657 | 291.42 | 63.66 |
| 29.7 | 3.00703 | 21.01 | 4.59 |
| 31.1 | 2.87542 | 261.58 | 57.14 |

### L-Tartaric Acid Form 2

To ca. 200 mg of the compound of Formula (I) Form 1 in a 20 mL screw cap vial, 2 mL of ethyl acetate was added to create a slurry. 1.05 equivalents of L-tartaric acid was added to the free base slurry as a slurry in 1.3 mL of ethyl acetate at ca. 20° C. Temperature cycling was carried out between ambient temperature (ca. 20° C.) and 50° C. in 4 hour cycles for ca. 2 days. Hold periods of 4 h at ambient temperature (ca. 20° C.) and 4 h hold periods at 50° C. were carried out. A sub-sample was analyzed by XRPD. The experiment was returned to temperature cycle for ca. 5 days then ca. 0.5 mL of the slurry was isolated by centrifugation (nylon, 0.22 μm) and analyzed by XRPD. The bulk slurry was isolated by filtration. The solids were dried under vacuum at ambient temperature (ca. 20° C.) for ca. 22 h to give Formula (I) L-tartaric acid co-crystal Form 2. XRPD, TGA/DSC, DSC, and DVS analysis were performed on Formula (I) L-tartaric acid co-crystal Form 2. The XRPD peak list is shown in Table 5 below.

An exemplary thermogravimetric analysis (TGA) graph of the L-tartaric acid co-crystal Form 2 is shown in FIG. 11.

An illustrative differential scanning calorimetry (DSC) graph of the L-tartaric acid co-crystal Form 2 is shown in FIG. 12. The L-tartaric acid co-crystal Form 2 is characterized by a differential scanning calorimetry (DSC) graph having an endothermic peak at about 158° C.

A dynamic vapor sorption (DVS) plot of the L-tartaric acid co-crystal Form 2 is shown in FIG. 13.

TABLE 5
XRPD peak list for Formula (I) L-Tartaric Acid Co-crystal Form 2

| Pos. [°2θ] | d-spacing [Å] | Height [cts] | Rel. Int. [%] |
|---|---|---|---|
| 3.3 | 26.88387 | 4718.3 | 97.64 |
| 6.6 | 13.3692 | 704.63 | 14.58 |
| 8.0 | 10.98033 | 1179.2 | 24.4 |
| 9.9 | 8.95147 | 1129.69 | 23.38 |
| 10.1 | 8.73818 | 290.6 | 6.01 |
| 11.2 | 7.92021 | 286.03 | 5.92 |
| 12.5 | 7.07266 | 1758 | 36.38 |
| 13.0 | 6.78757 | 581.83 | 12.04 |
| 13.3 | 6.6336 | 743.34 | 15.38 |
| 14.7 | 6.01682 | 498.01 | 10.31 |
| 15.0 | 5.91058 | 1622.71 | 33.58 |
| 15.2 | 5.83736 | 971.54 | 20.1 |
| 15.4 | 5.75345 | 415.21 | 8.59 |
| 16.1 | 5.4882 | 982.62 | 20.33 |
| 17.1 | 5.1934 | 1146.35 | 23.72 |
| 17.9 | 4.95316 | 4832.36 | 100 |
| 18.5 | 4.78233 | 1220.87 | 25.26 |
| 19.4 | 4.57684 | 1338.31 | 27.69 |
| 19.7 | 4.50655 | 932.27 | 19.29 |
| 20.3 | 4.36582 | 629.17 | 13.02 |
| 20.8 | 4.25705 | 281.63 | 5.83 |
| 21.3 | 4.165 | 631.24 | 13.06 |
| 21.7 | 4.10061 | 1530 | 31.66 |
| 22.3 | 3.97975 | 1350.75 | 27.95 |
| 23.6 | 3.7724 | 3971.61 | 82.19 |
| 24.4 | 3.64931 | 623.48 | 12.9 |
| 25.1 | 3.54013 | 2858.2 | 59.15 |
| 26.1 | 3.41661 | 490.59 | 10.15 |
| 26.3 | 3.38762 | 634.93 | 13.14 |
| 26.9 | 3.31292 | 1067.17 | 22.08 |
| 28.0 | 3.18712 | 611.74 | 12.66 |
| 28.3 | 3.1475 | 1205.67 | 24.95 |
| 30.2 | 2.95573 | 300.58 | 6.22 |
| 31.3 | 2.8565 | 341.19 | 7.06 |
| 31.7 | 2.81743 | 572.63 | 11.85 |
| 32.4 | 2.76327 | 127.02 | 2.63 |
| 32.8 | 2.72162 | 152.69 | 3.16 |
| 33.4 | 2.67809 | 183.96 | 3.81 |
| 33.9 | 2.64528 | 206.06 | 4.26 |

Single crystal growth procedure:

Ca. 20-50 mg of Formula (I) L-tartaric acid co-crystal Form 2 material was weighed into a 1.5 mL vial. Acetone was added in 100 μL aliquots until full dissolution of the solid was obtained. A needle was used to pierce the lid of the vial, and allow the acetone to slowly evaporate at ambient temperature. After three weeks, crystals of sufficient size for single-crystal X-ray diffraction analysis were obtained.

The asymmetric unit of Formula (I) L-tartaric acid co-crystal Form 2 contained two formula units of Formula (I):tartaric acid (1:1) and one water molecule. The tartaric acid groups are protonated confirming a co-crystal, hemi-hydrate was obtained. A summary of the unit cell parameters and refinement quality parameters for the single crystal is shown in Table 6 and Table 7.

TABLE 6
Unit cell parameters

| | |
|---|---|
| Monoclinic P2₁ | |
| a = 8.6993(2) Å | α = 90° |
| b = 11.9593(2) Å | β = 99.2210(11)° |
| c = 26.9060(5) Å | γ = 90° |
| Z = 4, Z′ = 2 | |
| V = 2763.06(9) Å³ | ρ = 1.516 g · cm⁻³ |

Data collection temperature = 100 K

TABLE 7
Refinement quality parameters

| | |
|---|---|
| R1(I > σ(I)) = 2.76% | S = 1.054 |
| wR2(all data) = 7.09% | Rint = 4.82% |

Flack parameter = −0.02 (4)

### L-Tartaric Acid Form 3

To a 100 mL vessel, 3.0 g Formula (I) compound was slurried in 18 mL THF at 65° C. for 30 minutes. Separately, L-tartaric acid (2.0 equivalents, 1.93 g) was mixed in 15 mL THF at 65° C. The tartaric acid was fully dissolved, and then added to the Formula (I) slurry.

After 30 minutes, 1 wt. % Formula (I) tartaric acid co-crystal Form 2 was added as seed and mixed at 50° C. for 8 hours, whereupon the mixture was cooled to 40° C. at 0.2° C./min and held for 30 minutes.

Heptane was added at 30 minute intervals. 3.7 mL of heptane was added as antisolvent to reach THF:heptane 90:10 v/v % (rate=1 volume/h) and held at temperature for 30 minutes. 4.6 mL of heptane was added as antisolvent to reach THF:heptane 80:20 v/v % (rate=1 volume/h) and held at temperature for 30 minutes. 5.9 mL of heptane was added as antisolvent to reach THF:heptane 70:30 v/v % (rate=1 volume/h) and held at temperature for 30 minutes.

The experiment was then cooled to 10° C. at 0.1° C./min and mixed for 16 hours.

The mixture was separated by vacuum filtration (Whatman GF/F filter paper). The solids were washed with 4 volumes of THF:heptane (50:50 v/v %) and placed in an oven to dry under vacuum at 40° C. for 16 hours to give Formula (I) tartaric acid co-crystal Form 3 by XRPD. Form 3 was determined to be 1:1 ratio of Formula (I): L-tartaric acid.

Upon standing at ambient temperature and humidity, Formula (I) L-tartaric acid co-crystal Form 3 converted to L-tartaric acid co-crystal Form 2.

TABLE 8
XRPD peak list for Formula (I) L-Tartaric Acid Co-crystal Form 3

| Pos. [°2θ] | d-spacing [Å] | Height [cts] | Rel. Int. [%] |
|---|---|---|---|
| 3.4 | 26.12146 | 3744.43 | 100 |
| 6.8 | 13.01846 | 668.95 | 17.87 |
| 8.1 | 10.85892 | 956.31 | 25.54 |
| 10.1 | 8.7911 | 1314.19 | 35.1 |
| 12.2 | 7.25747 | 656.45 | 17.53 |
| 12.6 | 7.01182 | 1498.8 | 40.03 |
| 14.8 | 5.96416 | 1038.1 | 27.72 |
| 15.2 | 5.81642 | 1670.73 | 44.62 |
| 15.6 | 5.68958 | 638.22 | 17.04 |
| 16.4 | 5.41439 | 2223.65 | 59.39 |
| 17.8 | 4.98353 | 840.86 | 22.46 |
| 18.1 | 4.90603 | 463.32 | 12.37 |
| 18.6 | 4.75782 | 526.46 | 14.06 |
| 19.6 | 4.5369 | 1373.62 | 36.68 |
| 20.2 | 4.38828 | 840.92 | 22.46 |
| 20.7 | 4.29168 | 1090.28 | 29.12 |
| 21.0 | 4.22961 | 544 | 14.53 |
| 21.7 | 4.08843 | 1389.1 | 37.1 |
| 23.0 | 3.86457 | 1614.15 | 43.11 |
| 24.6 | 3.62201 | 2695.09 | 71.98 |
| 25.3 | 3.51636 | 400.76 | 10.7 |
| 26.0 | 3.41872 | 928.23 | 24.79 |
| 28.1 | 3.1784 | 839.74 | 22.43 |
| 28.5 | 3.12542 | 584.11 | 15.6 |
| 29.8 | 2.99141 | 322.87 | 8.62 |
| 30.6 | 2.91795 | 461.74 | 12.33 |
| 32.1 | 2.79313 | 302.46 | 8.08 |

### L-Tartaric Acid Form 5

To a crystallization vessel, ca. 11 g Formula (I) compound was slurried in 49 mL acetone at 50° C. for 30 minutes. Separately, Ca. 1.25 equivalents (ca. 4.4 g) L-tartaric acid was dissolved in 110 mL acetone at 50° C. The acid solution was added to the Formula (I) slurry at 50° C. at 300 rpm. The mixture was held at 50° C. for 30 minutes and then cooled to 40° C. at 0.2° C./min and held for 45 minutes. The experimental mixture was polish filtered using a 400 mL polish filter with both Whatman grade 1 and GF/F filter papers. The clear filtered pale yellow solution was added into a crystallization vessel at 40° C.

The experiment was seeded using 2.0 wt. % co-crystal Pattern 2 and mixed at temperature for 25 minutes. The mixture was cooled to 30° C. at 0.2° C./min and held for 1.5 hours. 68 mL of heptane was added as antisolvent to reach acetone:heptane 70:30 v/v % (rate=0.1 mL/min) and held at temperature for 60 minutes. The experiment was cooled to 10° C. at 0.1° C./min and mixed for 16 hours to give a slurry. By XRPD a new pattern was observed, assigned as Form 5.

The experiment was separated by vacuum filtration (Whatman GF/F filter paper), washed with 4 volumes of acetone:heptane (50:50 v/v %) and placed in an oven to dry under vacuum at 40° C. for 16 hours.

TABLE 9
XRPD peak list for Formula (I) L-Tartaric Acid Co-crystal Form 5

| Pos. [°2θ] | d-spacing [Å] | Height [cts] | Rel. Int. [%] |
|---|---|---|---|
| 5.8 | 15.26795 | 8248.3 | 100 |
| 8.5 | 10.44233 | 2672 | 32.39 |
| 8.7 | 10.1766 | 2181.54 | 26.45 |
| 11.1 | 7.9494 | 580.74 | 7.04 |
| 11.5 | 7.68301 | 1522.69 | 18.46 |
| 12.1 | 7.31309 | 689.08 | 8.35 |
| 13.5 | 6.53375 | 3220.3 | 39.04 |
| 14.4 | 6.13105 | 1473.1 | 17.86 |
| 14.9 | 5.94848 | 2468.12 | 29.92 |
| 15.6 | 5.68504 | 262.69 | 3.18 |
| 16.3 | 5.4221 | 2088.92 | 25.33 |
| 16.6 | 5.32852 | 2479.94 | 30.07 |
| 17.0 | 5.21443 | 2510.1 | 30.43 |
| 17.2 | 5.16365 | 1100.76 | 13.35 |
| 17.4 | 5.07835 | 514.89 | 6.24 |
| 17.9 | 4.95464 | 1726.77 | 20.93 |
| 18.2 | 4.86525 | 695.38 | 8.43 |
| 18.8 | 4.72143 | 248.81 | 3.02 |
| 19.6 | 4.53479 | 2497.24 | 30.28 |
| 19.7 | 4.49853 | 1637.88 | 19.86 |
| 20.4 | 4.35726 | 2763.39 | 33.5 |
| 21.0 | 4.2223 | 448.11 | 5.43 |
| 21.4 | 4.1543 | 1642.03 | 19.91 |
| 21.6 | 4.10238 | 288.21 | 3.49 |
| 22.0 | 4.03733 | 725.06 | 8.79 |
| 22.2 | 3.99536 | 1570.94 | 19.05 |
| 22.6 | 3.93935 | 1041.21 | 12.62 |
| 22.9 | 3.87634 | 647.43 | 7.85 |
| 23.1 | 3.83979 | 1338.77 | 16.23 |
| 23.8 | 3.73213 | 480.34 | 5.82 |
| 24.4 | 3.64808 | 497.23 | 6.03 |
| 24.7 | 3.60249 | 445.2 | 5.4 |
| 25.1 | 3.53972 | 2968.87 | 35.99 |
| 25.5 | 3.49536 | 1200.09 | 14.55 |
| 25.7 | 3.46324 | 1255.2 | 15.22 |
| 26.1 | 3.41229 | 703.05 | 8.52 |
| 26.3 | 3.38402 | 588.92 | 7.14 |
| 27.0 | 3.30005 | 2954.99 | 35.83 |
| 27.5 | 3.24016 | 602.89 | 7.31 |
| 28.0 | 3.1888 | 261.5 | 3.17 |
| 28.7 | 3.10765 | 231.01 | 2.8 |
| 29.1 | 3.06998 | 661.16 | 8.02 |
| 29.7 | 3.00645 | 1377.07 | 16.7 |
| 30.7 | 2.90999 | 975.49 | 11.83 |
| 31.9 | 2.80242 | 141.17 | 1.71 |
| 32.6 | 2.74094 | 140.98 | 1.71 |
| 33.8 | 2.65241 | 290.86 | 3.53 |
| 34.4 | 2.60473 | 148.59 | 1.8 |

### Formula (I) Fumaric Acid Co-Crystal

To ca. 200 mg of Formula (I) Form B in a 20 mL screw cap vial, 2 mL of ethyl acetate was added to create a slurry. 1.05 equivalents of fumaric acid was added to the free base slurry as a slurry in 1.3 mL of ethyl acetate at ca. 20° C. Temperature cycling was carried out between ambient temperature (ca. 20° C.) and 50° C. in 4 hour cycles for ca. 2 days. Hold periods of 4 h at ambient temperature (ca. 20° C.) and 4 h hold periods at 50° C. were carried out. The vial was uncapped and allowed to dry at ambient temperature (ca. 20° C.) for ca. 24 h. The solids were then dried under vacuum at ambient temperature (ca. 20° C.) for ca. 18 h then analyzed by XRPD.

TABLE 10
XRPD peak list for Formula (I) Fumaric Acid Co-crystal

| Pos. [°2θ] | d-spacing [Å] | Height [cts] | Rel. Int. [%] |
|---|---|---|---|
| 3.5 | 24.9838 | 728 | 56.62 |
| 7.1 | 12.48955 | 908.81 | 70.69 |
| 7.4 | 12.01146 | 373.14 | 29.02 |
| 8.5 | 10.44032 | 131.78 | 10.25 |
| 9.3 | 9.55078 | 322.84 | 25.11 |
| 10.9 | 8.12277 | 159.62 | 12.42 |
| 14.2 | 6.22693 | 908.39 | 70.66 |
| 14.5 | 6.11743 | 1067.62 | 83.04 |
| 17.0 | 5.21503 | 684.87 | 53.27 |
| 19.2 | 4.6184 | 63.83 | 4.96 |
| 21.1 | 4.19839 | 273.95 | 21.31 |
| 22.7 | 3.92142 | 421.49 | 32.78 |
| 23.4 | 3.80016 | 596.43 | 46.39 |
| 23.9 | 3.71662 | 705.81 | 54.9 |
| 24.7 | 3.59777 | 1047.95 | 81.51 |
| 25.3 | 3.52189 | 1285.66 | 100 |
| 26.3 | 3.38854 | 409.18 | 31.83 |
| 27.8 | 3.21201 | 173.83 | 13.52 |
| 28.8 | 3.09615 | 317.71 | 24.71 |
| 29.6 | 3.01402 | 418.97 | 32.59 |
| 30.5 | 2.92466 | 78.07 | 6.07 |

### Formula (I) Hydrochloride Salt

2 mL of 2-Propanol was added to ca. 200 mg of CRD-4730 Pattern 1 in a 20 mL screw cap vial to create a slurry. 1.05 equivalents of HCl was added to the free base slurry as a stock solution in 1.3 mL of 2-propanol at ca. 20° C. Temperature cycling was carried out between ambient temperature (ca. 20° C.) and 50° C. in 4 hour cycles for ca. 2 days. Hold periods of 4 h at ambient temperature (ca. 20° C.) and 4 h hold periods at 50° C. were carried out. The vial was uncapped and allowed to evaporate at ambient temperature (ca. 20° C.) for ca. 24 h. The solids were then dried under vacuum at ambient temperature (ca. 20° C.) for ca. 18 h.

TABLE 11
XRPD peak list for Formula (I) Hydrochloride Salt

| Pos. [°2θ] | d-spacing [Å] | Height [cts] | Rel. Int. [%] |
|---|---|---|---|
| 4.1 | 21.52725 | 888.13 | 57.67 |
| 4.7 | 18.87943 | 211.24 | 13.72 |
| 7.6 | 11.63565 | 79.31 | 5.15 |
| 8.4 | 10.50631 | 764.82 | 49.67 |
| 9.6 | 9.16757 | 1304.32 | 84.7 |
| 11.1 | 7.96418 | 308.79 | 20.05 |
| 12.0 | 7.39934 | 147.4 | 9.57 |
| 12.4 | 7.11835 | 431.02 | 27.99 |
| 12.7 | 6.95061 | 1030.41 | 66.91 |
| 14.0 | 6.31626 | 147.98 | 9.61 |
| 14.5 | 6.11328 | 395.01 | 25.65 |
| 14.7 | 6.00841 | 578.22 | 37.55 |
| 15.3 | 5.7927 | 780.2 | 50.67 |
| 16.1 | 5.49375 | 371.65 | 24.13 |
| 16.9 | 5.23205 | 1366.31 | 88.73 |
| 18.0 | 4.9165 | 204.58 | 13.29 |
| 19.3 | 4.58506 | 526 | 34.16 |
| 20.8 | 4.26722 | 666.69 | 43.29 |
| 21.2 | 4.1803 | 1022.1 | 66.37 |
| 21.6 | 4.11443 | 803.54 | 52.18 |
| 22.8 | 3.90212 | 928.81 | 60.32 |
| 24.0 | 3.70742 | 1539.91 | 100 |
| 24.9 | 3.57727 | 512.79 | 33.3 |
| 25.2 | 3.52498 | 417.61 | 27.12 |
| 26.7 | 3.33794 | 814.5 | 52.89 |
| 28.2 | 3.16498 | 230.34 | 14.96 |
| 29.3 | 3.0441 | 357.73 | 23.23 |
| 30.0 | 2.98083 | 285.26 | 18.52 |
| 30.6 | 2.91517 | 202.42 | 13.15 |
| 31.5 | 2.83534 | 209.05 | 13.58 |
| 32.3 | 2.76979 | 122.82 | 7.98 |
| 33.3 | 2.68976 | 138.63 | 9 |
| 34.4 | 2.60177 | 55.48 | 3.6 |

### Formula (I) Methanesulfonate Salt

2 mL of Ethyl acetate was added to ca. 200 mg of Formula (I) Form B in a 20 mL screw cap vial to create a slurry. 1.05 equivalents of methanesulfonic acid were added to the free base slurry as a stock solution in 1.3 mL of ethyl acetate at ca. 20° C. Temperature cycling was carried out between ambient temperature (ca. 20° C.) and 50° C. in 4 hour cycles for ca. 2 days. Hold periods of 4 h at ambient temperature (ca. 20° C.) and 4 h hold periods at 50° C. were carried out. The solids were isolated by Büchner filtration with Grade 1 filter paper, and then were dried at 40° C. at ambient pressure.

TABLE 12
XRPD peak list for Formula (I) Methanesulfonate Salt

| Pos. [°2θ] | d-spacing [Å] | Height [cts] | Rel. Int. [%] |
|---|---|---|---|
| 3.7 | 23.6438 | 1795.07 | 39.51 |
| 7.5 | 11.77635 | 198.16 | 4.36 |
| 8.2 | 10.7873 | 642.12 | 14.13 |
| 10.0 | 8.83081 | 203.54 | 4.48 |
| 10.5 | 8.44483 | 384.86 | 8.47 |
| 10.9 | 8.14476 | 193.79 | 4.27 |
| 11.6 | 7.64667 | 192.27 | 4.23 |
| 12.2 | 7.27454 | 1088.12 | 23.95 |
| 12.4 | 7.14887 | 592.82 | 13.05 |
| 13.0 | 6.78457 | 780.82 | 17.19 |
| 14.6 | 6.05181 | 389.38 | 8.57 |
| 14.9 | 5.92926 | 670.16 | 14.75 |
| 15.1 | 5.85333 | 1255.43 | 27.63 |
| 15.8 | 5.60496 | 372.87 | 8.21 |
| 16.5 | 5.38015 | 2653.8 | 58.41 |
| 16.8 | 5.28045 | 820.62 | 18.06 |
| 17.4 | 5.08684 | 1522.06 | 33.5 |
| 17.6 | 5.041 | 1626.82 | 35.81 |
| 18.5 | 4.79144 | 735.22 | 16.18 |
| 18.9 | 4.69555 | 365.96 | 8.05 |
| 19.5 | 4.54177 | 689.41 | 15.17 |
| 20.3 | 4.38042 | 1586.56 | 34.92 |
| 20.8 | 4.26167 | 545.5 | 12.01 |
| 21.4 | 4.15291 | 2298.03 | 50.58 |
| 21.6 | 4.10741 | 1615.6 | 35.56 |
| 22.6 | 3.93153 | 1445.88 | 31.82 |
| 23.3 | 3.82055 | 842.46 | 18.54 |
| 23.9 | 3.71587 | 1335.24 | 29.39 |
| 24.4 | 3.64068 | 4543.32 | 100 |
| 25.1 | 3.542 | 969.92 | 21.35 |
| 25.6 | 3.47608 | 817.86 | 18 |
| 26.2 | 3.40312 | 730.81 | 16.09 |
| 26.6 | 3.33911 | 443.41 | 9.76 |
| 27.2 | 3.2724 | 248.36 | 5.47 |
| 27.6 | 3.23284 | 246.62 | 5.43 |
| 27.9 | 3.19143 | 301.98 | 6.65 |
| 28.2 | 3.15857 | 527.4 | 11.61 |
| 29.2 | 3.05813 | 362.11 | 7.97 |
| 29.6 | 3.02008 | 801.69 | 17.65 |
| 30.1 | 2.971 | 467.35 | 10.29 |
| 30.4 | 2.93656 | 332.54 | 7.32 |
| 31.2 | 2.86589 | 400.86 | 8.82 |
| 32.5 | 2.75379 | 207.27 | 4.56 |
| 33.1 | 2.70663 | 168.1 | 3.7 |
| 33.8 | 2.6468 | 166.27 | 3.66 |
| 34.3 | 2.61306 | 187.82 | 4.13 |

2 mL of ethyl acetate was added to ca. 200 mg of Formula (I) Form B in a 20 mL screw cap vial to create a slurry. 1.05 equivalents of phosphoric acid was added to the free base slurry as a stock solution in 1.3 mL of ethyl acetate at ca. 20° C. Temperature cycling was carried out between ambient temperature (ca. 20° C.) and 50° C. in 4 hour cycles for ca. 2 days. Hold periods of 4 h at ambient temperature (ca. 20° C.) and 4 h hold periods at 50° C. were carried out. The solids were isolated by Büchner filtration with Grade 1 filter paper, and then were dried at 40° C. at ambient pressure.

TABLE 13
XRPD peak list for Formula (I) Phosphoric Acid Co-Crystal

| Pos. [°2θ] | d-spacing [Å] | Height [cts] | Rel. Int. [%] |
|---|---|---|---|
| 3.7 | 24.17656 | 4149.27 | 100 |
| 8.1 | 10.87217 | 279.79 | 6.74 |
| 10.3 | 8.56421 | 199.31 | 4.8 |
| 11.2 | 7.88106 | 126.7 | 3.05 |
| 12.2 | 7.26363 | 189.5 | 4.57 |
| 12.7 | 6.9577 | 114.6 | 2.76 |
| 13.4 | 6.61939 | 342.1 | 8.24 |
| 14.2 | 6.2179 | 158.58 | 3.82 |
| 14.7 | 6.01588 | 171.63 | 4.14 |
| 15.0 | 5.91765 | 128.29 | 3.09 |
| 15.3 | 5.79431 | 130.83 | 3.15 |
| 16.3 | 5.44557 | 798.21 | 19.24 |
| 16.5 | 5.37961 | 1146.98 | 27.64 |
| 17.6 | 5.0396 | 552.86 | 13.32 |
| 19.1 | 4.63155 | 186.28 | 4.49 |
| 19.8 | 4.4778 | 526.21 | 12.68 |
| 20.7 | 4.29604 | 250.94 | 6.05 |
| 21.3 | 4.17747 | 402.69 | 9.71 |
| 21.7 | 4.09425 | 206.21 | 4.97 |
| 22.2 | 3.99959 | 411.91 | 9.93 |
| 22.6 | 3.93707 | 510.46 | 12.3 |
| 22.9 | 3.87829 | 1046.4 | 25.22 |
| 23.4 | 3.8007 | 344.58 | 8.3 |
| 23.6 | 3.77121 | 467.22 | 11.26 |
| 24.5 | 3.62803 | 582.7 | 14.04 |
| 25.1 | 3.54359 | 304.4 | 7.34 |
| 25.8 | 3.45688 | 253.91 | 6.12 |
| 26.2 | 3.40174 | 143.72 | 3.46 |
| 27.1 | 3.29348 | 314.84 | 7.59 |
| 27.8 | 3.20726 | 43.82 | 1.06 |
| 28.4 | 3.13848 | 276.72 | 6.67 |
| 28.7 | 3.10397 | 47.87 | 1.15 |
| 29.6 | 3.01725 | 102.46 | 2.47 |
| 30.5 | 2.92509 | 94.28 | 2.27 |
| 32.2 | 2.77694 | 45.28 | 1.09 |
| 33.3 | 2.69105 | 71.13 | 1.71 |

### Example 3. Stability of a Crystalline Form and Co-Crystal of Formula (I)

### Competitive Stability of Free Base Polymorphs of Formula (I)

Competitive slurrying was carried out to identify which of Formula (I) Form A, Form B, or Form C was the most stable free base form. Competitive slurries were carried out at 20° C. and 60° C. using the following procedure: 1.5 mL slurries of Formula (I) were prepared in t-BME, acetone, ethanol, and 2-Me THF at 60° C. in 2 mL screw cap vials. The slurries were syringe filtered (0.45 μm PTFE filter) after ca. 1 hour to obtain saturated Formula (I) solutions. Ca. 0.6 mL of saturated solution was added to ca. 10 mg of free base Forms A, B, and C to form slurries. The experiments were agitated at either ambient temperature (ca. 20° C.) and 60° C. for 48 h.

After ca. 24 h the acetone slurries were observed to be immobile and 100 μL of acetone was added to the ca. 20° C. experiment, and 200 μL of acetone was added to the 60° C. experiment. After ca. 40 h the 60° C. t-BME experiment was observed to contain no solvent and 600 μL of t-BME was added.

After 48 h the slurries were isolated by centrifugation (nylon, 0.22 μm) and analyzed by XRPD. Where mixtures were observed competitive slurrying was continued until mixtures were no longer observed (21 days). In all solvents tested, Formula (I) Form C was the most stable polymorph (Table 14).

TABLE 14
Competitive Slurry Experiments

| | | Time until Form C was only | |
|---|---|---|---|
| temperature | solvent | detected polymorph | |
| ambient (ca. 20° C.) | t-BME | 21 | days |
| | acetone | 2 | days |
| | Ethanol | 8 | days |
| | 2-Me THF | 9 | days |
| 60° C. | t-BME | 15 | days |
| | acetone | 2 | days |
| | Ethanol | 2 | days |
| | 2-Me THF | 2 | days |

### Form C

The 7 day stability results are shown in Table 15. XRPD analysis showed Formula (I) Form C was retained under all conditions. HPLC analysis indicated no loss in purity under all conditions.

TABLE 15
Seven Day Stability for Formula (I) Form C

| | | Input Purity | HPLC Purity |
|---|---|---|---|
| Condition | XRPD Analysis | (% area) | (% area) |
| 40° C./75% RH | Form C | 98.8 | 99.0 |
| 25° C./60% RH | Form C | | 99.0 |
| 80° C. | Form C | | 98.9 |

Formula (I) Form C was evaluated for stability (Table 16 and Table 17). The Form C was stored in polyethylene double bags sealed with Nylon lock ties, and inside a capped polyethylene container at Wilmington PharmaTech Company. The storage conditions were long-term condition 25° C./60% RH, or accelerated condition 40° C./75% RH. The samples were analyzed for appearance, assay, related substances, chiral purity, water content and crystallinity.

TABLE 16
Stability for Formula (I) Form C at 25° C./60% RH

| | Acceptance | Time (Months) | | |
|---|---|---|---|---|
| Test | Criterion | 0 | 12 | 24 |
| Description | Report | Off-white | Off-white | Off-white |
| | Result | powder | powder | powder |
| Chemical Purity | 96.0-103.0% | 100.1 | 99.4 | 99.4 |
| Assay (wt %) | (anhydrous) | | | |
| Related | | | | |
| Substances (%): | | | | |
| Individual Impurities | ≤0.5% each | | | |
| Impurity #1 | | 0.05 | 0.05 | 0.05 |
| Impurity #2 | | 0.18 | 0.18 | 0.18 |
| Total Impurities (%) | ≤2.0% | 0.23 | 0.23 | 0.23 |
| Water (%) | Report | 0.29 | 0.32 | 0.35 |
| | Results | | | |
| Chiral Purity (area %) | ≥99.0% | >99.9 | >99.9 | >99.9 |
| Crystallinity | Conforms | Conforms | Conforms | Conforms |
| | to Form C | to Form C | to Form C | to Form C |

NT = not tested at time point.

TABLE 17
Stability for Formula (I) Form C at 40° C./75% RH

| | Acceptance | Time (Months) | | |
|---|---|---|---|---|
| Test | Criterion | 0 | 6 | 12 |
| Description | Report | Off-white | Off-white | Off-white |
| | Result | powder | powder | powder |
| Chemical Purity | 96.0-103.0% | 99.6 | 99.3 | 99.6 |
| Assay (wt %) | (anhydrous) | | | |
| Related | | | | |
| Substances (%): | | | | |
| A. Individual | ≤0.5% each | | | |
| Impurities | | | | |
| Impurity #1 | | 0.09 | 0.08 | 0.08 |
| Impurity #2 | | 0.19 | 0.19 | 0.19 |
| Impurity #3 | | <0.05 | 0.05 | <0.05 |
| B. Unidentified | ≤0.2% each | 0.07 | 0.06 | 0.06 |
| Impurities | | | | |
| (RRT: %) | | 0.05 | 0.06 | |
| Total Impurities (%) | ≤2.0% | 0.40 | 0.44 | 0.33 |
| Water (%) | Report | 0.36 | 0.15 | 0.49 |
| | Results | | | |
| Chiral Purity (area %) | ≥99.0% | >99.9 | >99.9 | >99.9 |
| Crystallinity | Conforms | Conforms | Conforms | Conforms |
| | to Form C | to Form C | to Form C | to Form C |

### L-Tartaric Acid Form 2

Formula (I) L-tartaric acid Form 2 co-crystal was evaluated for stability (Table 18 and Table 19). The co-crystal was stored in polyethylene double bags sealed with Nylon lock ties with two (2) 0.5 g silica gel desiccant packets in between, and inside a capped polyethylene container containing four (4) 33 g Silica Gel desiccant packs. The storage conditions were long-term condition 25° C./60% RH or accelerated condition 40° C./75% RH. The samples were analyzed for appearance, assay, related substances, chiral purity, water content and crystallinity.

TABLE 18
Stability for Formula (I) L-Tartaric Acid Form 2 at 25° C./60% RH

| | Acceptance | Time (Months) | | |
|---|---|---|---|---|
| Test | Criterion | 0 | 3 | 6 |
| Description | Report | Off-white | Off-white | Off-white |
| | Result | powder | powder | powder |
| Chemical Purity | 96.0-103.0% | 99.3 | 99.6 | 99.3 |
| Assay (wt %) | (ASFB) | | | |
| Related | | | | |
| Substances (%)^{a}: | | | | |
| Individual Impurities | ≤0.5% each | | | |
| Impurity #1 | | 0.25 | 0.25 | 0.26 |
| Total Impurities (%) | ≤3.0% | 0.25 | 0.25 | 0.26 |
| Water (%) | Report | 1.59 | 1.58 | 1.55 |
| | Results | | | |
| Chiral Purity (area %) | ≥99.0% | >99.9 | NT | >99.9 |
| Crystallinity | Conforms | Conforms | Conforms | Conforms |
| | to Form C | to Form C | to Form 2 | to Form 2 |

NT = not tested at time point.

TABLE 19
Stability for Formula (I) L-Tartaric Acid Form 2 at 40° C./75% RH

| | Acceptance | Time (Months) | | |
|---|---|---|---|---|
| Test | Criterion | 0 | 3 | 6 |
| Description | Report | Off-white | Off-white | Off-white |
| | Result | powder | powder | powder |
| Chemical Purity | 96.0-103.0% | 99.3 | 99.3 | 100.0 |
| Assay (wt %) | (ASFB) | | | |
| Related | | | | |
| Substances (%)^{a}: | | | | |
| Individual Impurities | ≤0.5% each | | | |
| Impurity #1 | | 0.25 | 0.25 | 0.25 |
| Total Impurities (%) | ≤3.0% | 0.25 | 0.25 | 0.25 |
| Water (%) | Report | 1.59 | 1.56 | 1.60 |
| | Results | | | |
| Chiral Purity (area %) | ≥99.0% | >99.9 | NT | >99.9 |
| Crystallinity | Conforms to | Conforms | Conforms | Conforms |
| | Form 2 | to Form 2 | to Form 2 | to Form 2 |

NT = not tested at time point.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A crystalline form of the compound of Formula (I): 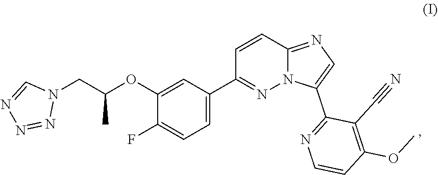 [SMILES: COc1ccnc(-c2cnc3ccc(-c4ccc(F)c(O[C@@H](C)Cn5cnnn5)c4)nn23)c1C#N]
or a pharmaceutically acceptable salt thereof.

2.-3. (canceled)

4. The crystalline form of claim 1, which is Form A.

5. The crystalline form of claim 4, wherein the Form A is characterized by an X-ray powder diffraction (XRPD) pattern comprising peaks at 4.7, 16.1, and 26.5 degrees 2θ (±0.2 degrees 2θ), wherein the XRPD is made using CuK_{α1} radiation.

6.-7. (canceled)

8. The crystalline form of claim 1, which is Form B.

9. The crystalline form of claim 8, wherein the Form B is characterized by an X-ray powder diffraction (XRPD) pattern comprising peaks at 16.1, 16.9, and 26.6 degrees 2θ (±0.2 degrees 2θ), wherein the XRPD is made using CuK_{α1} radiation.

10. The crystalline form of claim 1, which is Form C.

11. The crystalline form of claim 10, wherein the Form C is characterized by an X-ray powder diffraction (XRPD) pattern comprising peaks at 14.4, 22.2, and 25.2 degrees 2θ (±0.2 degrees 2θ), wherein the XRPD is made using CuK_{α1} radiation.

12.-13. (canceled)

14. The crystalline form of claim 1, which is a co-crystal of the compound of Formula (I).

15. (canceled)

16. The crystalline form of claim 14, which is L-tartaric acid co-crystal Form 5.

17. The crystalline form of claim 16, wherein the L-tartaric acid co-crystal Form 5 is characterized by an X-ray powder diffraction (XRPD) pattern comprising peaks at 5.8, 8.5, and 13.5 degrees 2θ (±0.2 degrees 2θ), wherein the XRPD is made using CuK_{α1} radiation.

18. (canceled)

19. The crystalline form of claim 14, which is L-tartaric acid co-crystal Form 1.

20. The crystalline form of claim 19, wherein the L-tartaric acid co-crystal Form 1 is characterized by an X-ray powder diffraction (XRPD) pattern comprising peaks at 25.5, 28.1, and 29.1 degrees 2θ (0.2 degrees 2θ), wherein the XRPD is made using CuK_{α1} radiation.

21. The crystalline form of claim 14, which is L-tartaric acid co-crystal Form 2.

22. The crystalline form of claim 21, wherein the L-tartaric acid co-crystal Form 2 is characterized by an X-ray powder diffraction (XRPD) pattern comprising peaks at 3.3, 17.9, and 23.6 degrees 2θ (±0.2 degrees 2θ), wherein the XRPD is made using CuK_{α1} radiation.

23. (canceled)

24. The crystalline form of claim 14, which is L-tartaric acid co-crystal Form 3.

25. (canceled)

26. The crystalline form of claim 14, which is a fumaric acid co-crystal.

27. (canceled)

28. The crystalline form of claim 1, which is a hydrochloride.

29. (canceled)

30. The crystalline form of claim 1, which is a methanesulfonate.

31. (canceled)

32. The crystalline form of claim 1, which is a phosphoric acid co-crystal.

33. (canceled)

34. A pharmaceutical composition comprising a crystalline form of claim 1, and a pharmaceutically acceptable excipient.

35. A method of preparing Form C of the compound of Formula (I), comprising:
(a) warming a mixture of the compound of Formula (I) and a solvent comprising acetone, water, dichloromethane, or methyl ethyl ketone, or a combination thereof, and
(b) cooling the mixture,
thereby preparing the Form C of the compound of Formula (I).

36. A method of preventing or treating a CaMKII associated disease or condition, comprising administering a therapeutically effective amount of a crystalline form of claim 1.
